(19) United States
(12) Patent Application Publication
(10) Pub. No.: US 2015/0017732 A1
(43) Pub. Date: Jan. 15, 2015

(54) COLORIMETRIC METHOD TO DETECT ILLICIT DRUGS

(71) Applicants: Tsunghsueh Wu, Platteville, WI (US); Charles Cornett, Platteville, WI (US)

(72) Inventors: Tsunghsueh Wu, Platteville, WI (US); Charles Cornett, Platteville, WI (US)

(21) Appl. No.: 14/329,714

(22) Filed: Jul. 11, 2014

Related U.S. Application Data
(60) Provisional application No. 61/937,356, filed on Feb. 7, 2014.
(60) Provisional application No. 61/845,632, filed on Jul. 12, 2013.

(51) Int. Cl. G01N 33/94; G01N 21/78

(52) U.S. Cl. CPC G01N 33/94; G01N 33/946; G01N 33/948; G01N 33/9486; G01N 21/78
USPC 436/92; 436/96; 436/98; 422/430; 436/111; 436/93

(57) ABSTRACT

A colorimetric assay to detect certain compounds and a kit therefore are provided.

### CROSS-REFERENCE TO RELATED APPLICATIONS

This application claims the benefit of the filing date of U.S. application Ser. No. 61/937,356, filed on Feb. 7, 2014, and U.S. application Ser. No. 61/845,632, filed on Jul. 12, 2013, the disclosures of which are incorporated by reference herein.

### BACKGROUND

Color tests have been used by criminalists for decades to provide a rapid, inexpensive means of determining if an unknown compound merits further investigation. Law enforcement officers rely upon commercially produced, presumptive color tests to determine probable cause for arrest and subsequent substance identification by crime laboratory personnel and techniques. Following a positive field presumptive test, the evidence (a potential controlled substance) is sent to the crime laboratory for further processing. This involves an indicative test and finally a confirmatory test. Color tests may also be used by crime laboratory personnel in certain applications, however, the confirmatory test is an instrumental assay, typically by gas chromatograph-mass spectrometry.

### SUMMARY OF THE INVENTION

The invention provides a colorimetric assay to detect a piperazine, an indoline, an indole, an azabicyclo, a morphinan, or an amine containing compound. The assay includes providing a test sample suspected of having a piperazine, indoline, indole, azabicyclo, morphinan, or amine containing compound including but not limited to synthetic cathinones and synthetic cannabinoids, and reagents including an aqueous buffer, an organic solvent, and a sulfonic acid, sultone or oxothian-2,2,dioxide containing dye and optionally a Dragendorff's reagent. The reagents may be provided in a single receptacle, e.g., an ampoule, or in individual receptacles which are combined, to form a composition having the buffer, the organic solvent and the dye. The sample and the reagents are combined and optionally agitated to provide a mixture. Then it is determined whether the mixture with the test sample has a different color than a control mixture that lacks the test sample. In one embodiment, the test sample is a solid, e.g., in the form of a powder. In one embodiment, the test sample is in liquid form. In one embodiment, if the test sample has a different color, the intensity of the color may detected. In one embodiment, the Dragendorff's reagent, e.g., a reagent containing a mixture of bismuth nitrate, glacial acetic acid potassium iodide and water. In one embodiment, the dye is a synthetic dye, e.g., one for laboratory use or a food grade dye. In one embodiment, the dye is a natural dye, e.g., one for laboratory use or a food grade dye. In one embodiment, the dye is methyl orange, xylenol orange, calmagite, FD&C blue 1, metanil yellow, 1-(2-hydroxyl-1-naphthylazo)-2-naphthol-4-sulfonic acid zinc salt, 3-((E)-(4-((E)-(4-amino-7-sulfonatonaphthalen-1-yl)diazenyl)-7-sulfonatonaphthalen-1-yl)diazenyl)naphthalene-1,5-disulfonate sodium salt, 2-((4-hydroxyphenyl)(4-oxocyclohexa-2,5-dien-1-ylidene)methyl)benzenesulfonate sodium salt, or 4-((E)-(4-(ethylamino)-3-methylphenyl)((E)-4-(ethyliminio)cyclohexa-2,5-dien-1-ylidene)methyl)-3-sulfobenzenesulfonate sodium salt. In one embodiment, the buffer has a pH of about 0 to 8, e.g., 2 to 8. In one embodiment, the buffer has a pH of about 4, e.g., a pH that is from 3.5 to 4.5. In one embodiment, the buffer is a phosphate, sodium acetate, citrate, phosphate-citrate, tartrate, or acetic acid buffer. In one embodiment, the mixture is compared to a negative control, e.g., which has the buffer, the organic solvent and the dye but lacks a piperazine, indoline, indole, azabicyclo, morphinan, or amine containing compound, and/or a positive control sample, such as one having the buffer, the organic solvent, the dye and at least one of a piperazine, indoline, indole, azabicyclo, morphinan, or amine containing compound. In one embodiment, after combining the test sample and the buffer, organic solvent and dye, the organic phase is yellow, blue, pink, or red. In one embodiment the positive control sample includes at least one of a benzylpiperazine (BZP), trifluoromethyl phenylpiperazine (TFMPP), methamphetamine, 3-methylenedioxy-methamphetamine (MDMA), cocaine, a synthetic cathinone, or a synthetic cannabinoid. In one embodiment, the buffer, organic solvent and dye are in a single receptacle prior to contact with the test sample. In one embodiment, the receptacle is formed of a synthetic material. In one embodiment, the receptacle is formed of a plastic. In one embodiment, the receptacle is formed of glass. In one embodiment, the receptacle is formed of a translucent material. In one embodiment, the first receptacle is present in or can be placed in a larger receptacle that has a mechanism for sealing. In one embodiment, the first or the larger receptacle has a white background for visualization of results. In one embodiment, the composition or reagents have a volume less than 3 mL. In one embodiment, the composition or reagents have a volume greater than about 0.5 mL.

Also provided is a kit comprising a first receptacle having a composition comprising a buffer, an organic solvent, and a sulfonic acid, sultone or oxothian-2,2,dioxide containing dye. In one embodiment, the dye is methyl orange, xylenol orange, calmagite, metanil yellow, 1-(2-hydroxyl-1-naphthylazo)-2-naphthol-4-sulfonic acid zinc salt, 1-(2-hydroxyl-1-naphthylazo)-2-naphthol-4-sulfonic acid zinc salt, 3-((E)-(4-((E)-(4-amino-7-sulfonatonaphthalen-1-yl)diazenyl)-7-sulfonatonaphthalen-1-yl)diazenyl)naphthalene-1,5-disulfonate sodium salt, 2-((4-hydroxyphenyl)(4-oxocyclohexa-2,5-dien-1-ylidene)methyl)benzenesulfonate sodium salt, or 4-((E)-(4-(ethylamino)-3-methylphenyl)((E)-4-(ethyliminio)cyclohexa-2,5-dien-1-ylidene)methyl)-3-sulfobenzenesulfonate sodium salt. In one embodiment, the kit further comprises one or more other receptacles, e.g., a second receptacle having a composition comprising the buffer, the organic solvent, a sulfonic acid, sultone or oxothian-2,2,dioxide containing dye, a dye other than a sulfonic acid, sultone or oxothian-2,2,dioxide containing dye or other test reagent, e.g., the Marquis test or Scott's reagent. In one embodiment, the buffer has a pH of about 2 to 5. In one embodiment, the buffer has a pH of about 0 to 8, e.g., 2 to 7 or 8. In one embodiment, the buffer is a phosphate, sodium acetate, citrate, phosphate-citrate or acetic acid buffer. In one embodiment, the receptacle is formed of a synthetic material. In one embodiment, the receptacle is formed of a plastic. In one embodiment, the receptacle is formed of a glass. In one embodiment, the organic solvent is chloroform, toluene, dimethylformamide or dimethylsulfoxide. In one embodiment, the organic solvent is chloroform, toluene or xylene. In one embodiment, the buffer is a phosphate, sodium, citrate, tartrate, phosphate-citrate or acetic acid buffer. The receptacle may be formed of a material that is resistant to degradation by the reagents. A kit may contain written instructions and optionally a color chart, e.g., to determine the amount of the compound that is detected.

The dyes useful in the methods or kits of the invention are present in amounts useful to detect the compounds in amounts from about 0.001 mg to about 30 mg, e.g., from about 0.001 mg to about 0.01 mg, about 0.01 mg to about 0.1 mg, about 0.1 mg to about 20 mg, about 1 mg to about 3 mg and up to about 10 mg. The kits may be used under a variety of environmental conditions, including at temperatures from about −3° C. to about 40° C.

For example, a test sample is added to one compartment in a receptacle having two compartments, where the second compartment has the reagents described herein. Then the receptacle is subjected to pressure to mix the components in the two compartments.

Also provided is a series of assays or kits for sequential screening, e.g., in the same receptacle. In one embodiment, the kit further comprises an additional receptacle having one or more reagents to conduct an additional colorimetric test, e.g., to eliminate or reduce false positives. The kit may thus include reagents for any of the following tests/reagents: Liebermann, Marquis, Chen-Kao (Chen), Simon, Duquenois-Levine, Scott, Mecke, iodoform, 2,4-dinitrophenylhydrazine, or Schiff's reagent, and/or tests measuring the presence of azabicyclo groups, morphinan groups, amine groups and carbonyl groups. For example, one receptable may have methyl orange and another may have a reagent for the Marquis test. The majority of synthetic cathinones or naturally occurring cathinones, when mixed with methyl orange, will result in a yellow color and, when mixed with a reagent for the Marquis test, will show no reaction or a yellow color depending on the molecular structure of the cathinone.

### BRIEF DESCRIPTION OF THE FIGURES

FIG. 1. The results of the presumptive test using methyl orange. The right ampoule contains benzylpiperazine while the left ampoule is a control experiment.

FIG. 2. The results of a presumptive test using 1-(2-hydroxyl-1-naphthylazo)-2-naphthol-4-sulfonic acid zinc salt. The right ampoule is the control experiment (no drug) and the left ampoule contains drug (BZP).

FIG. 3. Detection of synthetic cannabinoids and alkaloids. Vial #1 shows the orange precipitate (ppt) formation after mixing JWH018 with Dragendorff's reagent. Vial #2 is the Dragendorff's reagent in water where no precipitate is present. Vial #3 is the Dragendorff's reagent in methanol (showing no precipitate). Vial #4 shows the ppt formation after adding JWH018 to Dragendorff's reagent-modified methyl orange test (DMO test). Vial #5 shows the result of the control DMO test when no JWH018 is present.

FIG. 4. Depiction of an exemplary kit having a pouch that contains instructions and one or more receptacles having a composition comprising a dye.

FIG. 5. Depiction of exemplary receptacles having a composition comprising a dye and a color code for determining whether a test sample has reacted or interacted with the dye.

FIGS. 6A-W. Summary of results for exemplary test compounds. The concentration of dye used was 0.1% in aqueous solution. The concentration of bismuth nitrate was 2 mM and the concentration of potassium iodide was 0.15 M in Dragendorff's reagent.

FIGS. 7A-E. The results of a test for 4 different synthetic cathinones with methyl orange.

FIGS. 8A-E. The results of a test for 4 different synthetic cathinones with Dragendorff's reagent.

FIGS. 9A-BB. Results for different drugs tested at pH2, pH 4 or pH 7.

FIGS. 10A-B. Results with two reagents including Scott's test (cobalt thiocyanate).

FIG. 11. Results with cocaine and Benadryl. Benadryl (50) and cocaine (309) were subject to the same reagents and conditions and the color was recorded after each addition. The end result is the modified Scott's test. Cocaine and benadryl show different final results, and so these reagents differentiate between the two substances.

### DETAILED DESCRIPTION

Color tests have been used in forensic analysis for decades to preliminarily screen samples for drugs of abuse. When combined with certain chemical reagents, many substances produce a clear, distinct and predictable color. The color reactions are typically not specific to one compound, but are produced by many compounds within a drug class or by unrelated substances containing a common functional or structural group. It is important to note that aspects of color reactions have never been fully explained due to the frequent occurrence of anomalous responses. Unexpected color results and the inherent subjectivity of color interpretation emphasize the presumptive nature of color tests. Their purpose is to indicate the possible presence or absence of certain substances in a sample. If unexpected or ambiguous results are observed from a color test, the sample should be subjected to confirmatory instrumental analysis for identification.

Current color based presumptive drug tests are summarized in Table 1. The tests mentioned in Table 1 have specificity for the targeted drugs.

TABLE 1

| Test | Chemicals | Positive Results |
|---|---|---|
| Dillie- | Cobalt thiocyanate, methanol, | Barbiturates: violet |
| Koppanyi | and isopropylamine | |
| Simon's | Sodium nitroprusside and | Methamphetamine: deep |
| | sodium biocarbonate | blue |
| Marquis | Formaldehyde and Sulfuric | Opium based drugs will |
| | Acid | give a purple color |
| | | Amphetamines will turn |
| | | it Orange/brown color |
| Mecke | Selenous acid and sulfuric | Ectasy (MDMA) will |
| | acid | give a purple color |
| | | Heroin will give a |
| | | green color after |
| | | prolonged agitation |
| Scotts | Cobalt thiocynate, distilled | Cocaine will turn the |
| Test | water, glycerin, hydrochloric | liquid blue |
| | acid and chloroform | |
| Duquenois- | Vanillin, acetaldehyde, ethyl | Marijuana will turn |
| Levine | alcohol, and chloroform | the solution purple |
| PDMAB | P-dimethylaminobenzldeyde, | LSD will turn blue- |
| | hydrochloric acid and ethyl | purple |
| | alcohol | |
| Cobalt Acetate | Cobalt acetate, methanol and | Barbiturates will turn |
| | isopropylamine | violet-blue |

In contrast, the colorimetric assay disclosed herein can provide a single presumptive test for a wide variety of drugs. Thus, an investigator needs just one test instead of multiple tests to determine probable cause for an arrest for a potential controlled substance. The assay is thus a rapid color test for use in the field. For example, an investigator can place the questioned substance in a disposable ampoule of chemical reagents necessary for the presumptive identification of controlled substances including but not limited to benzylpiperazine (BZP), 3-trifluoromethyl-phenyl-piperaine (TFMPP), methamphetamine (Meth), MDMA (Ectasy), cocaine, a synthetic cathinone, and/or synthetic cannabinoids.

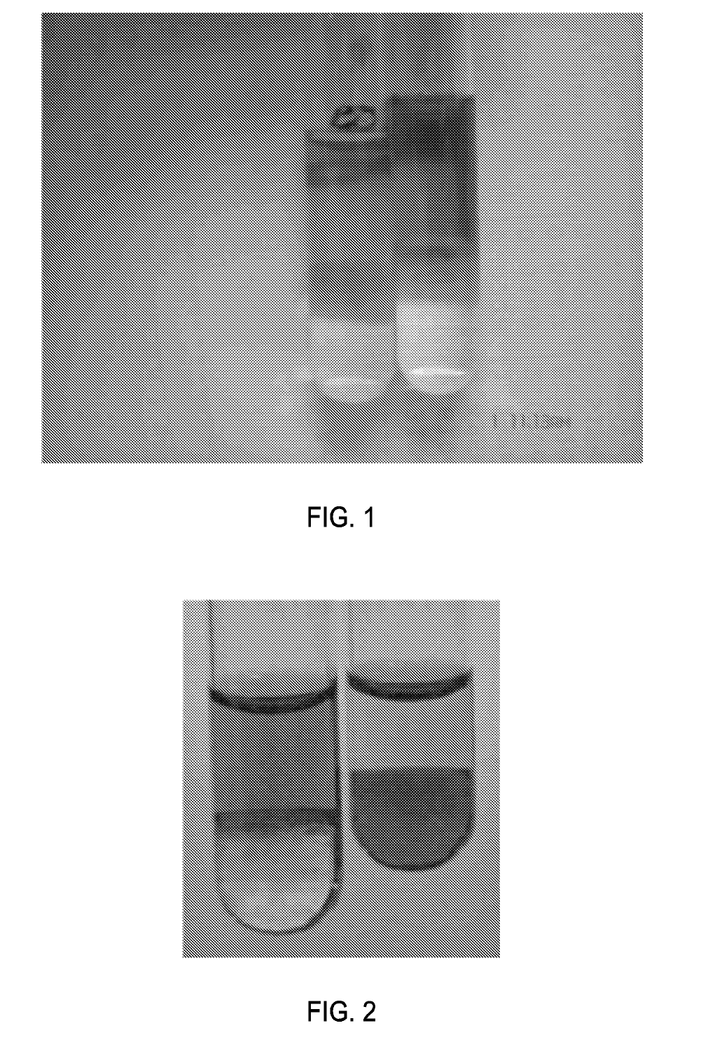

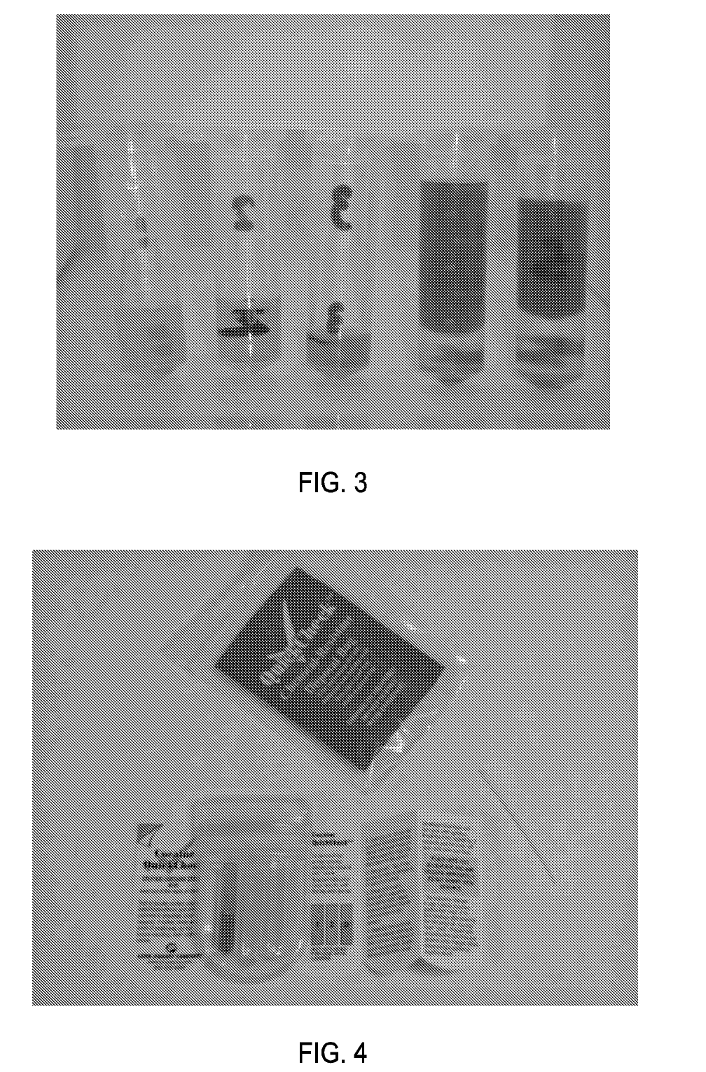

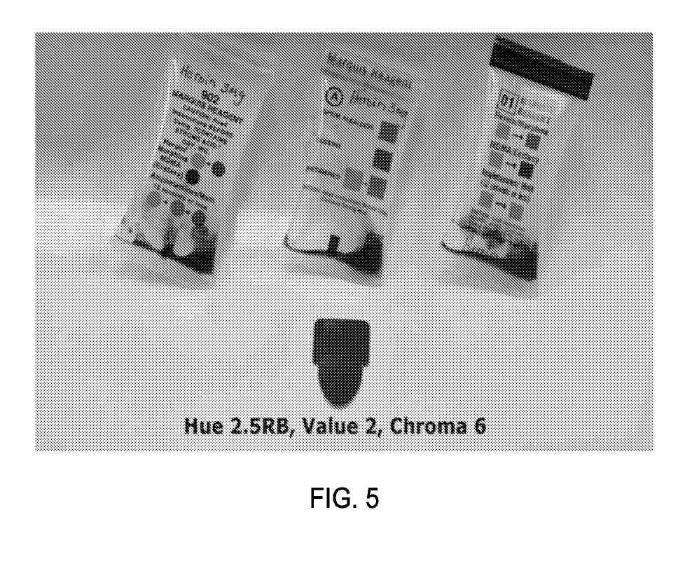

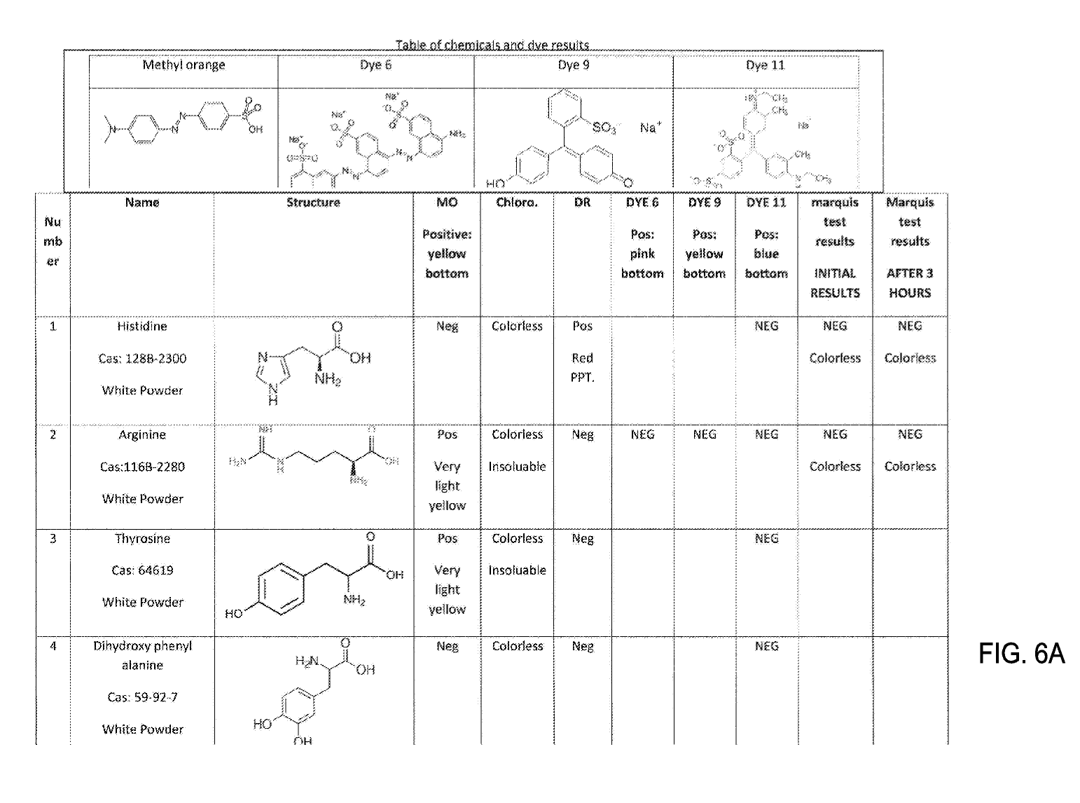

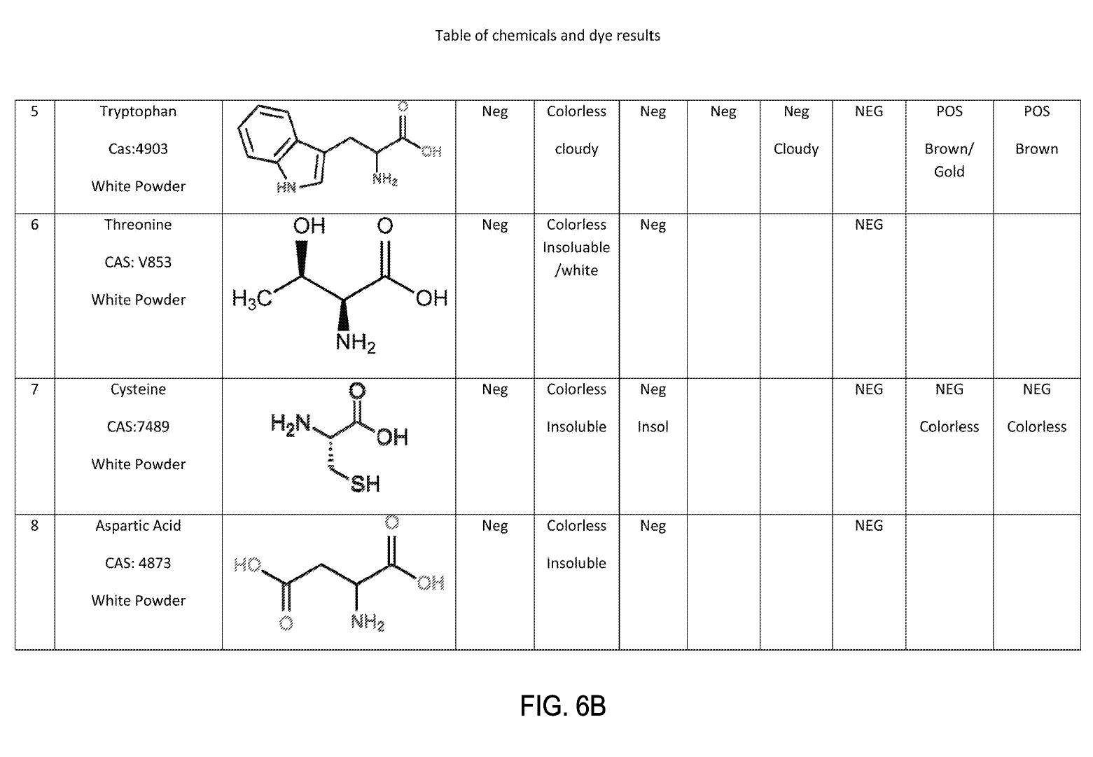

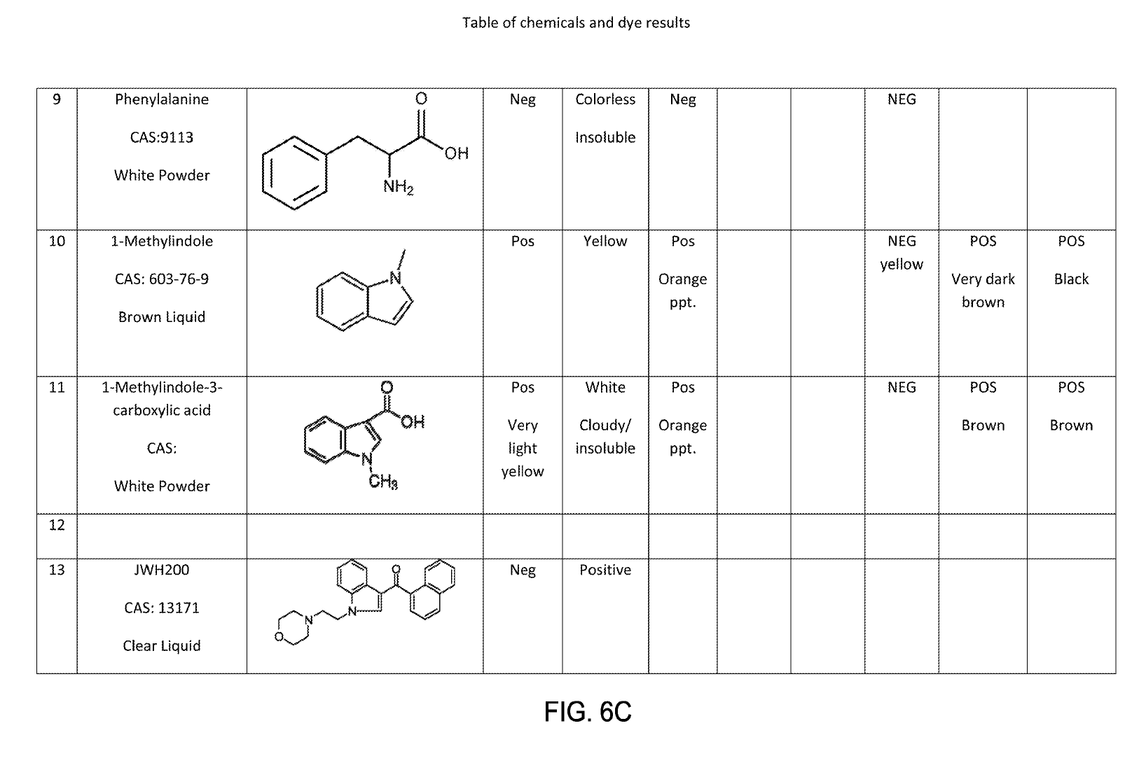

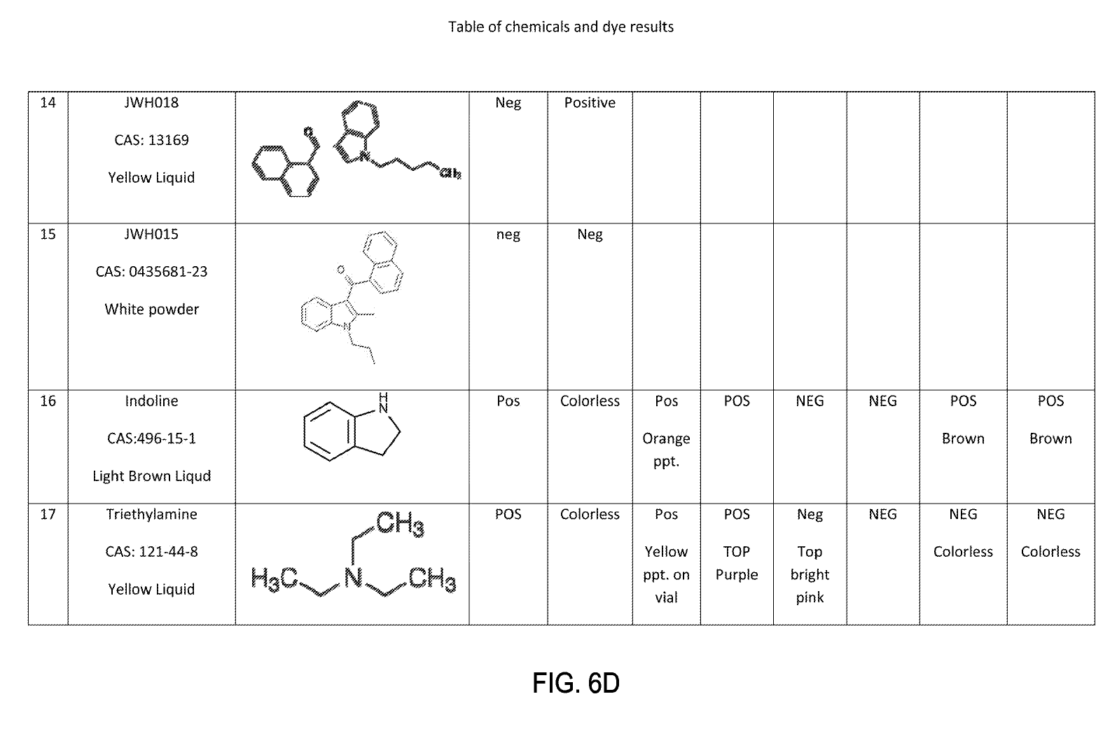

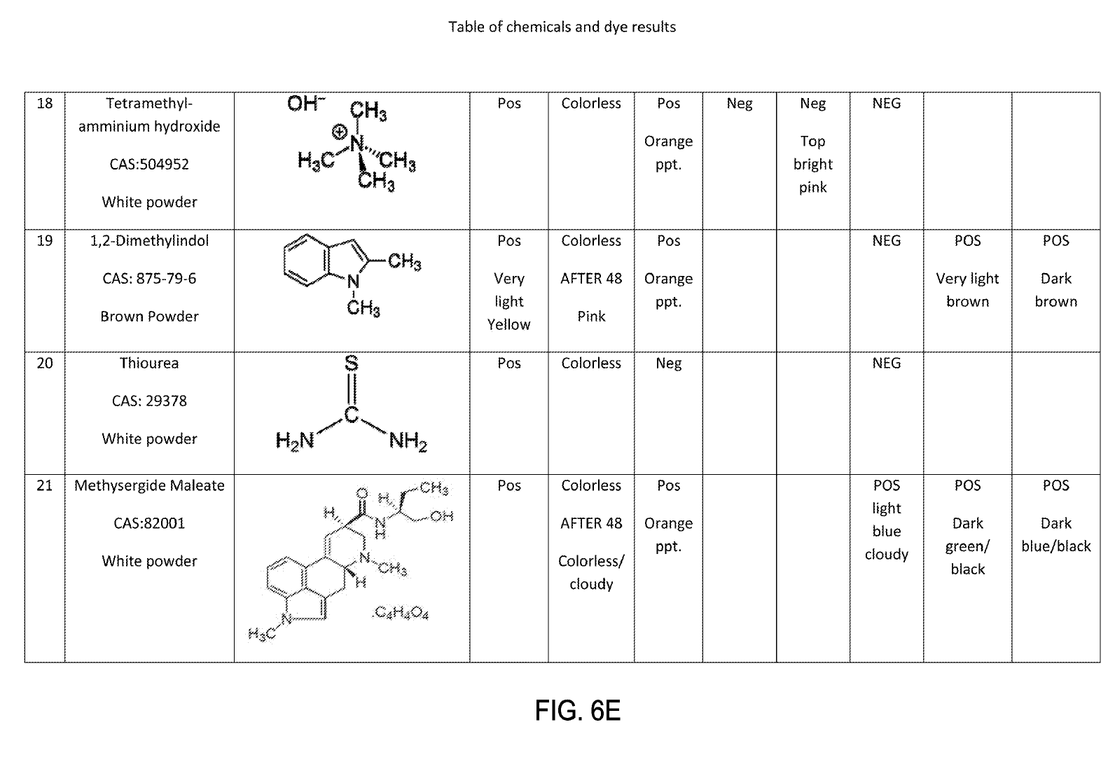

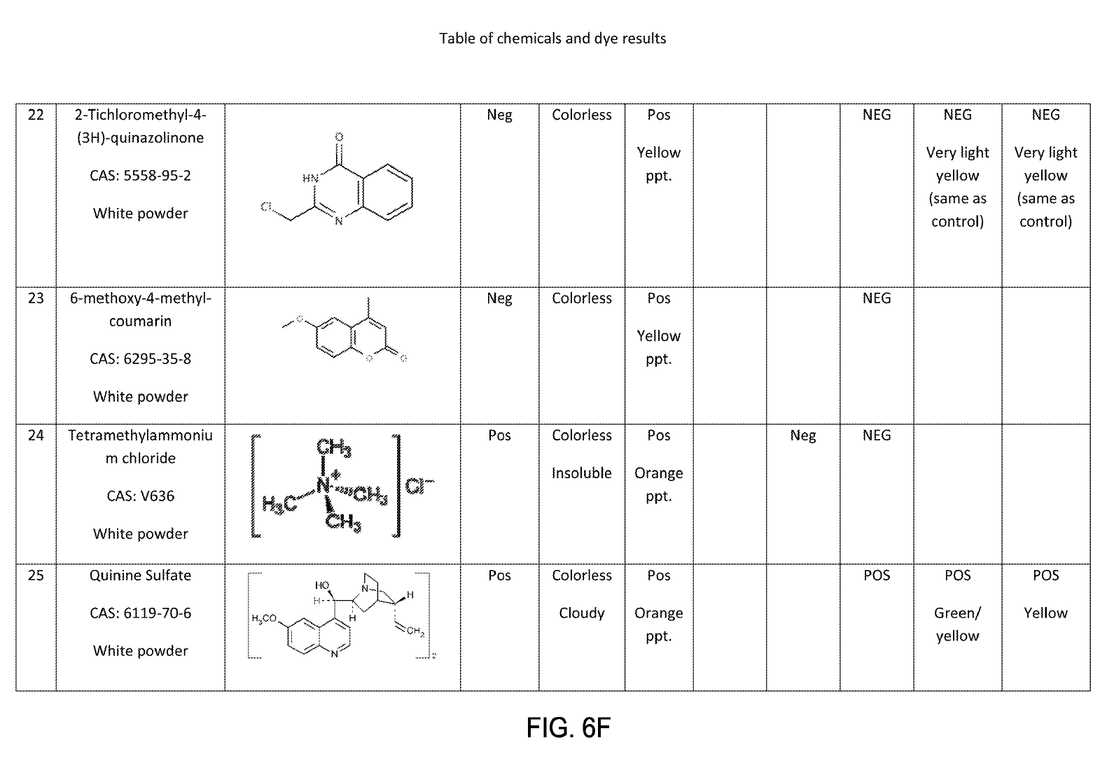

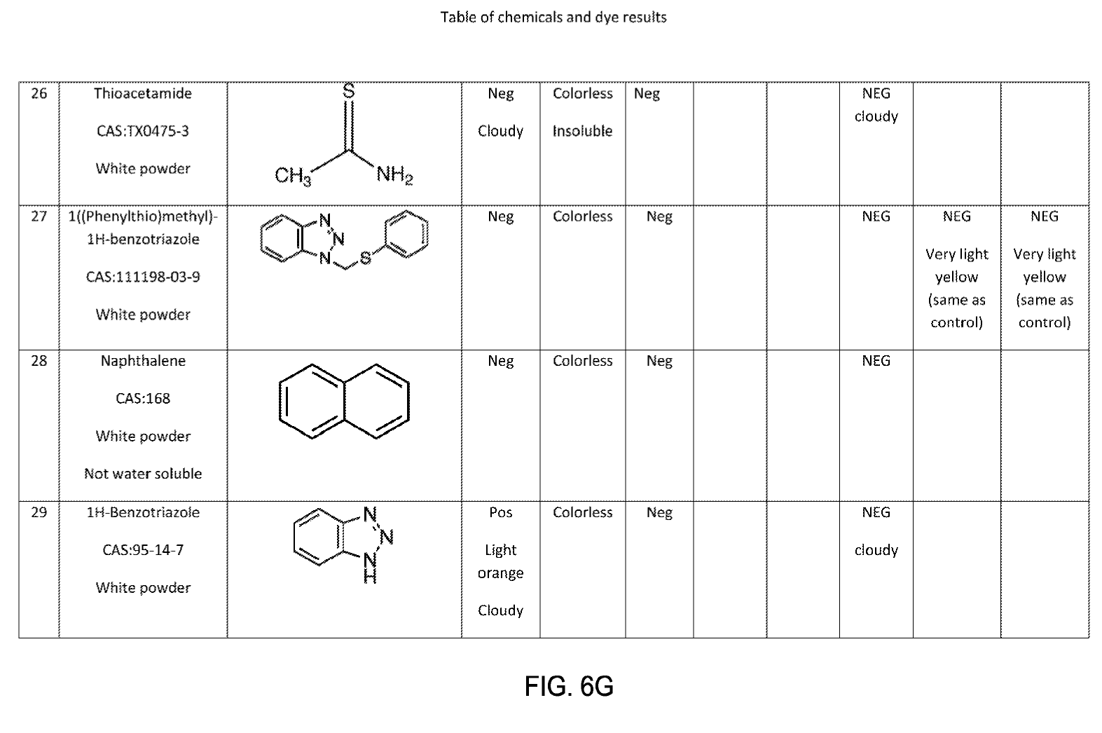

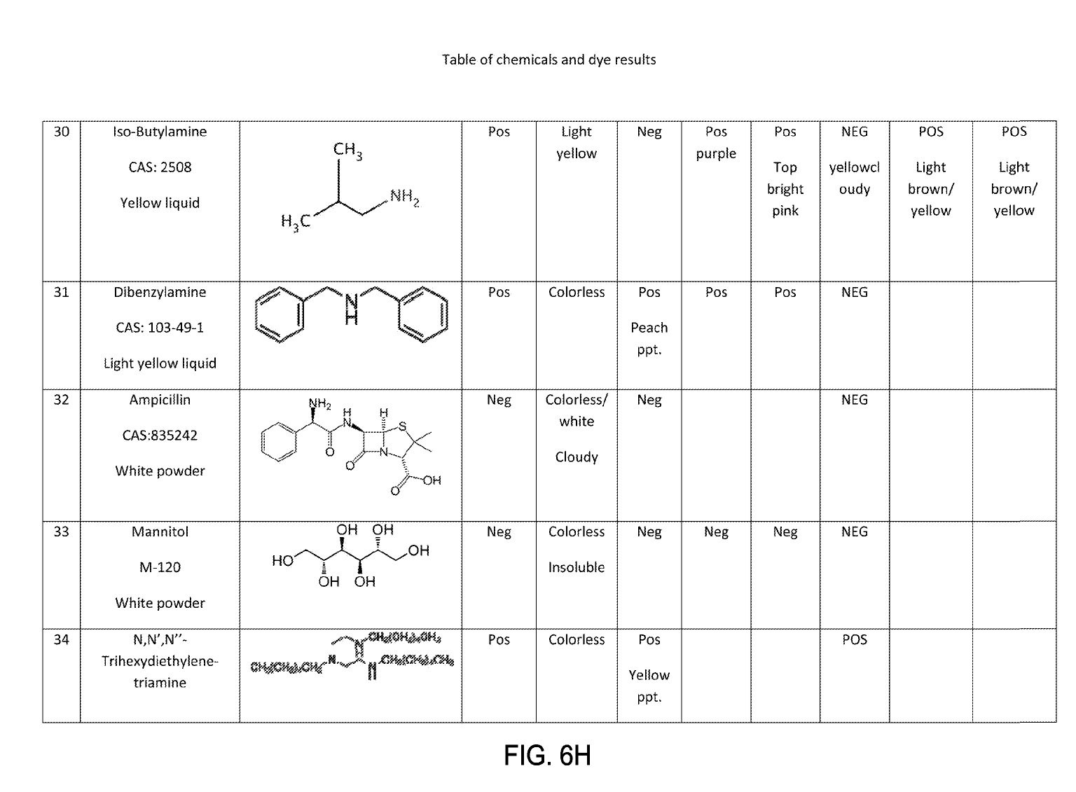

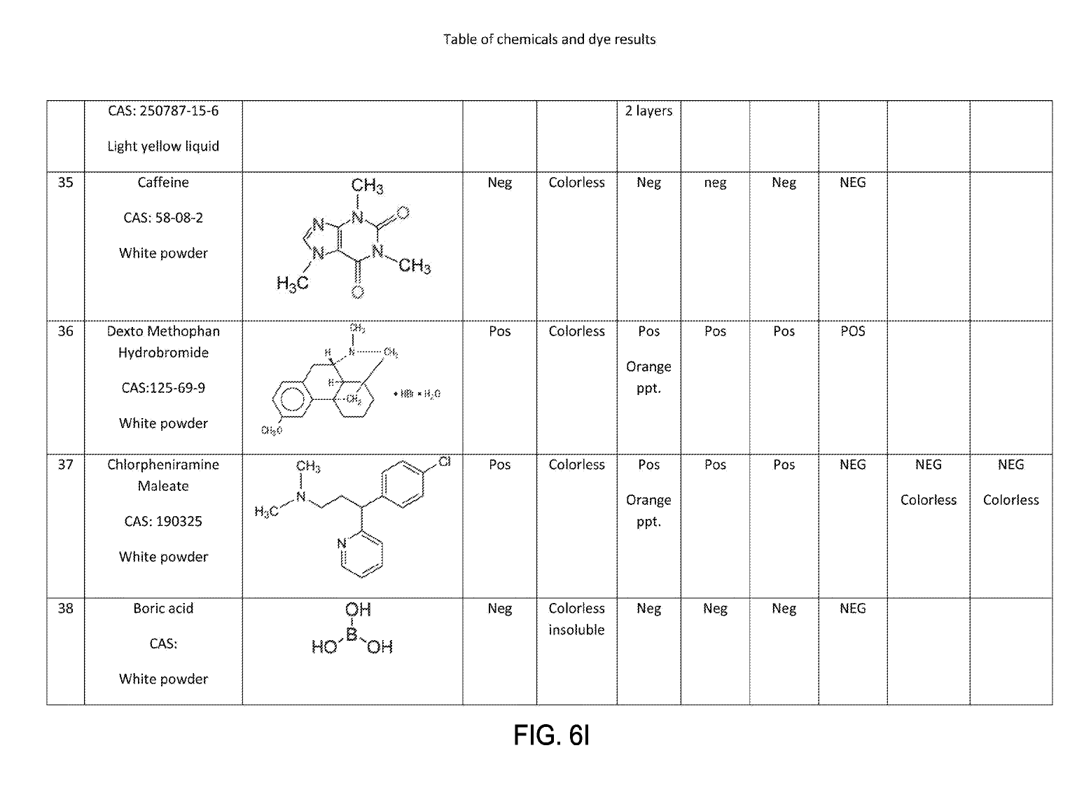

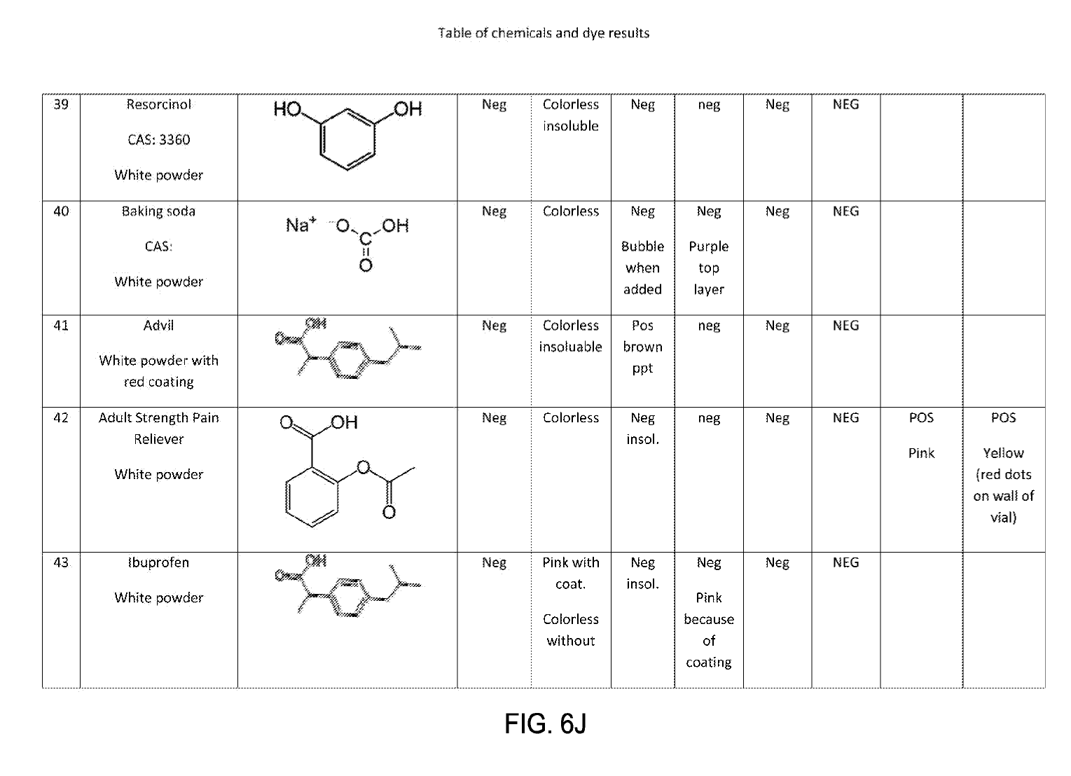

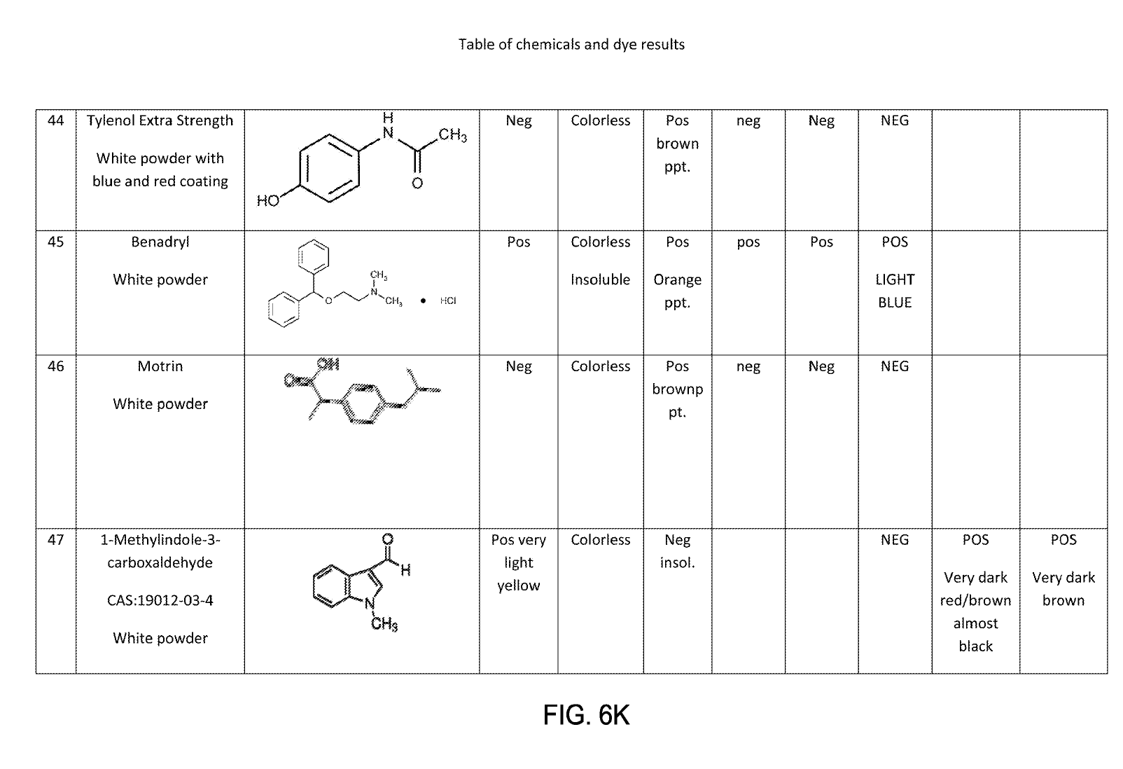

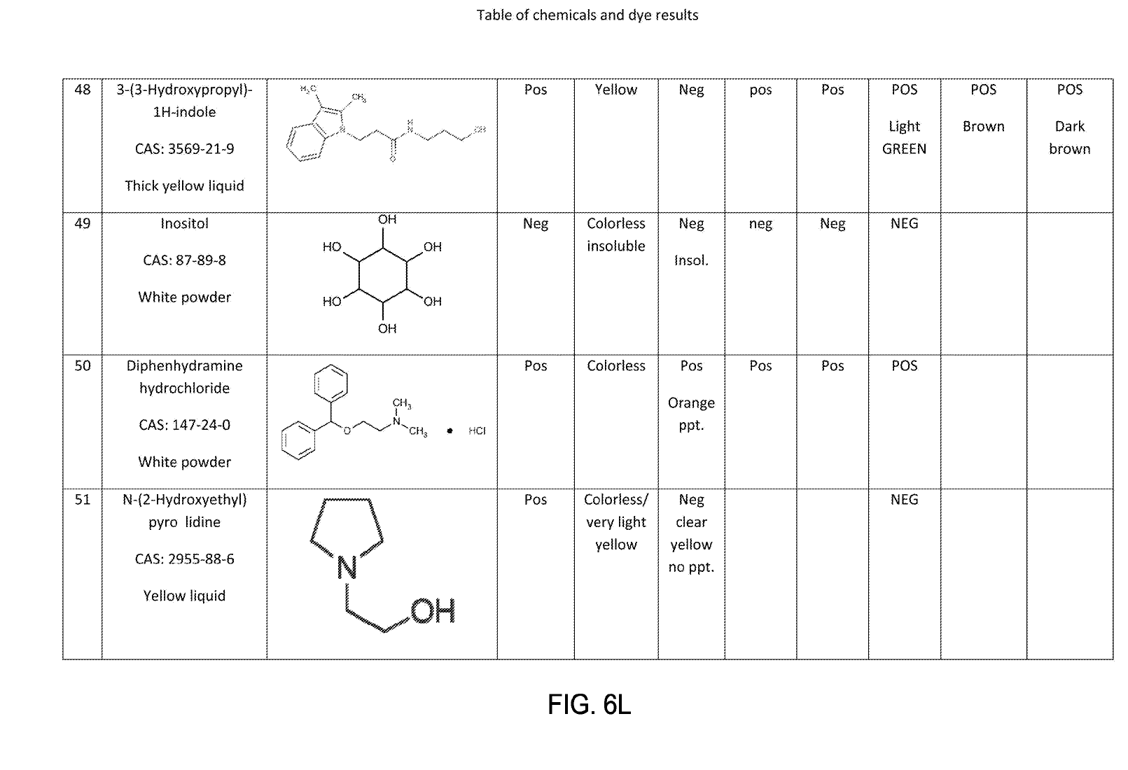

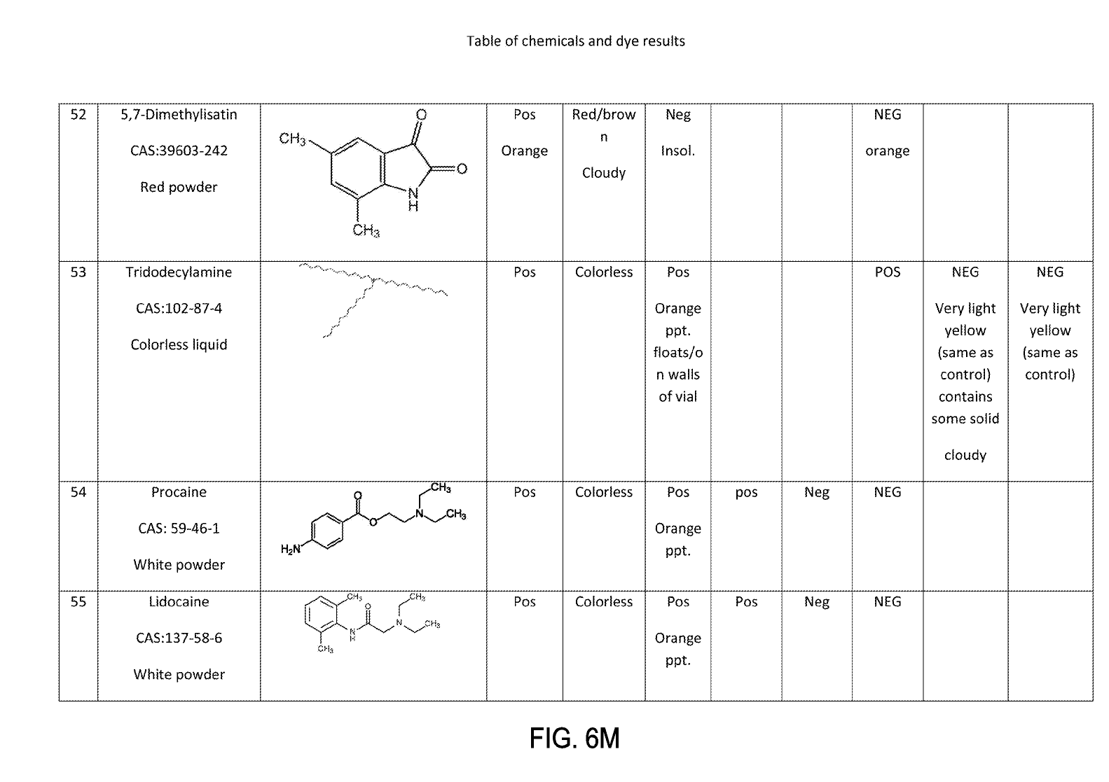

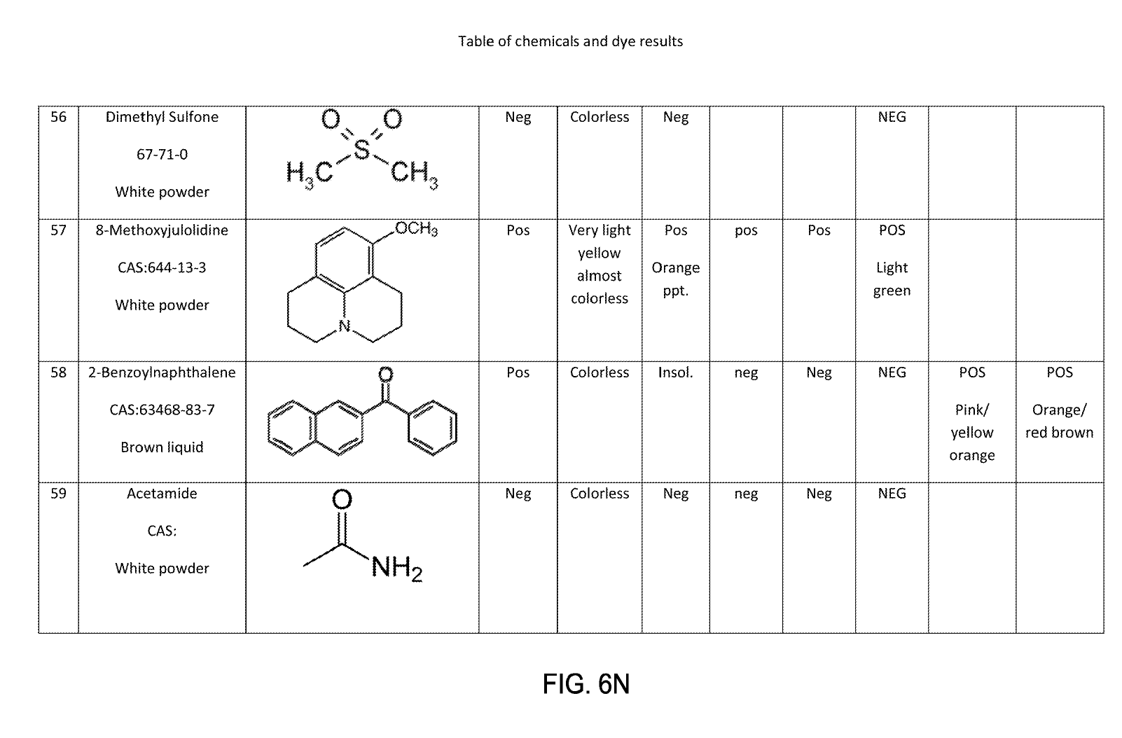

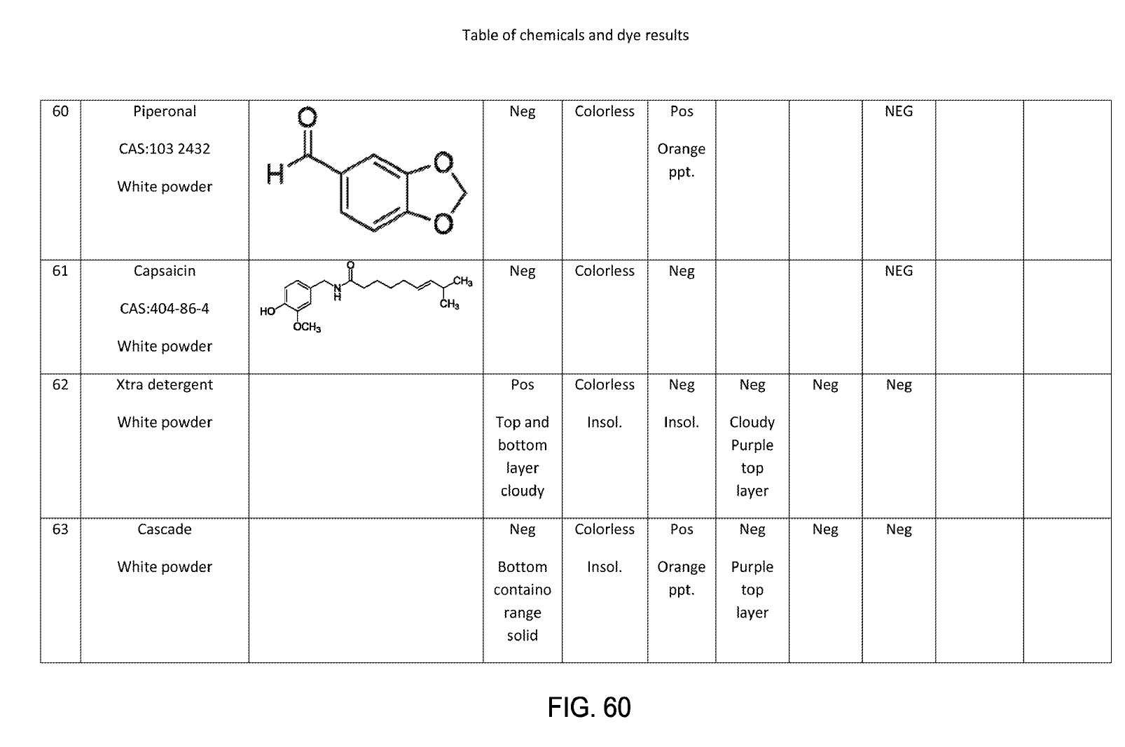

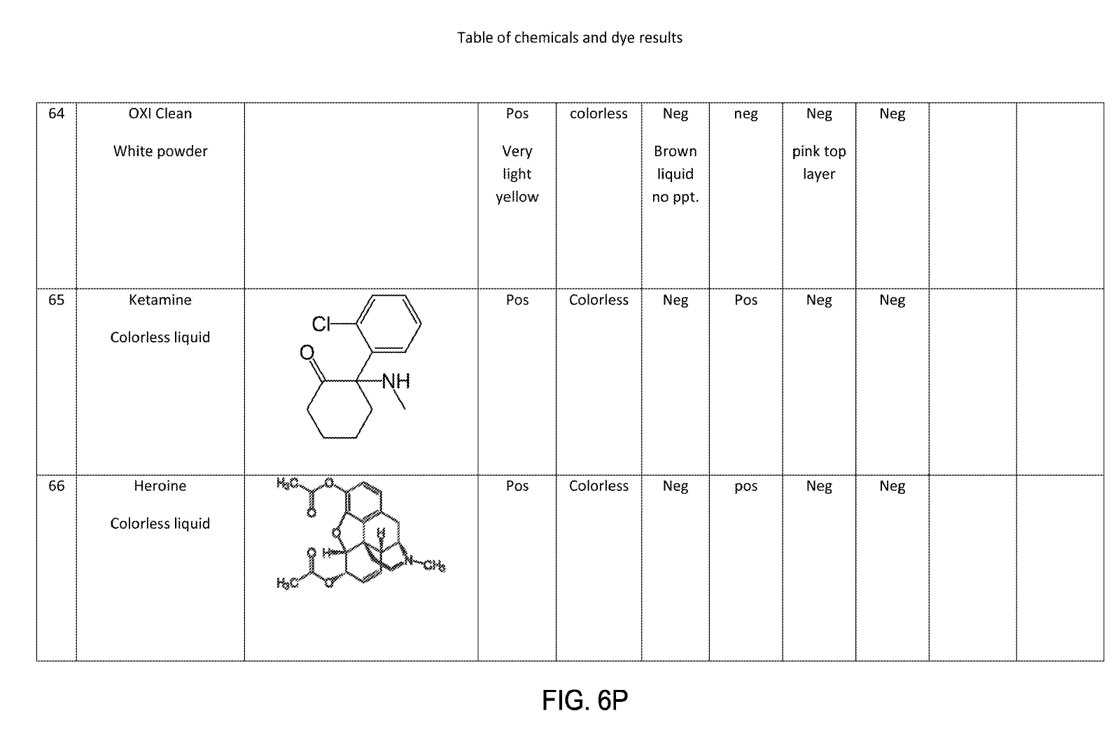

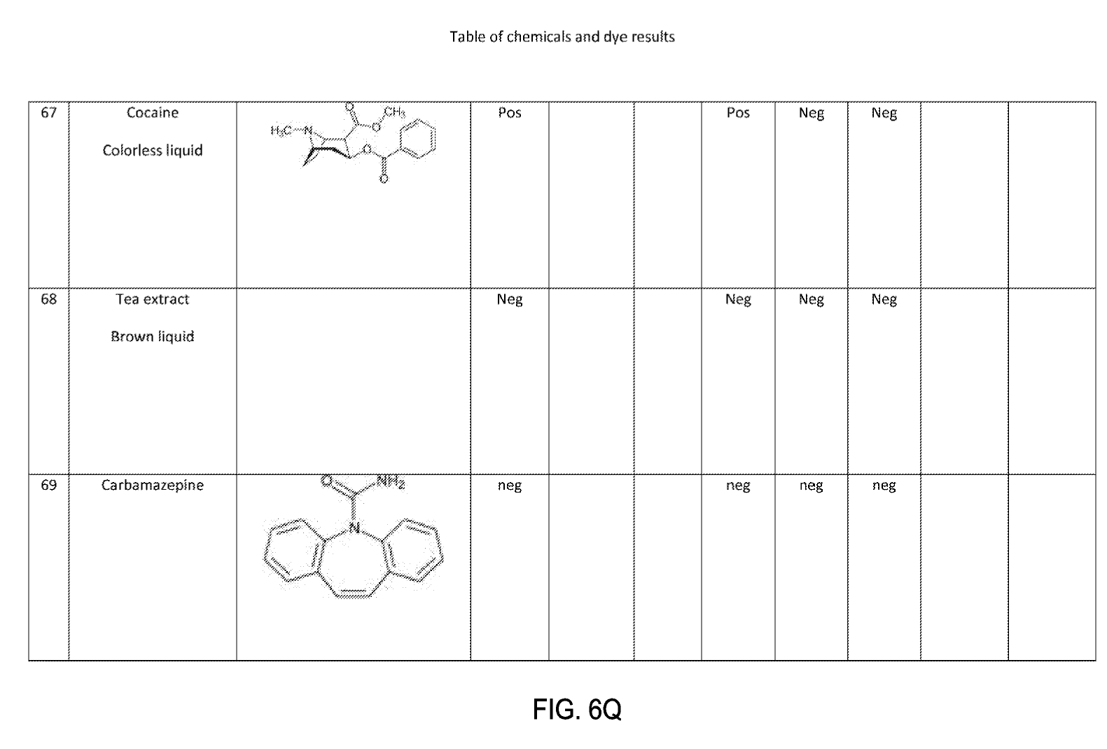

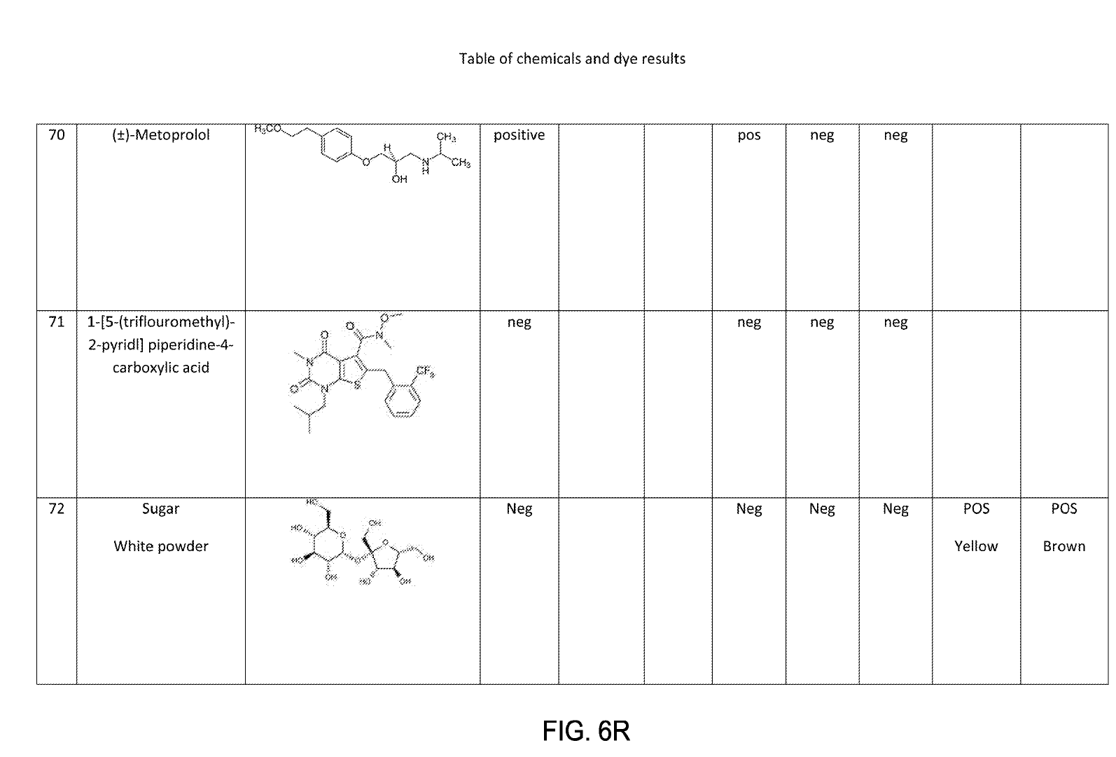

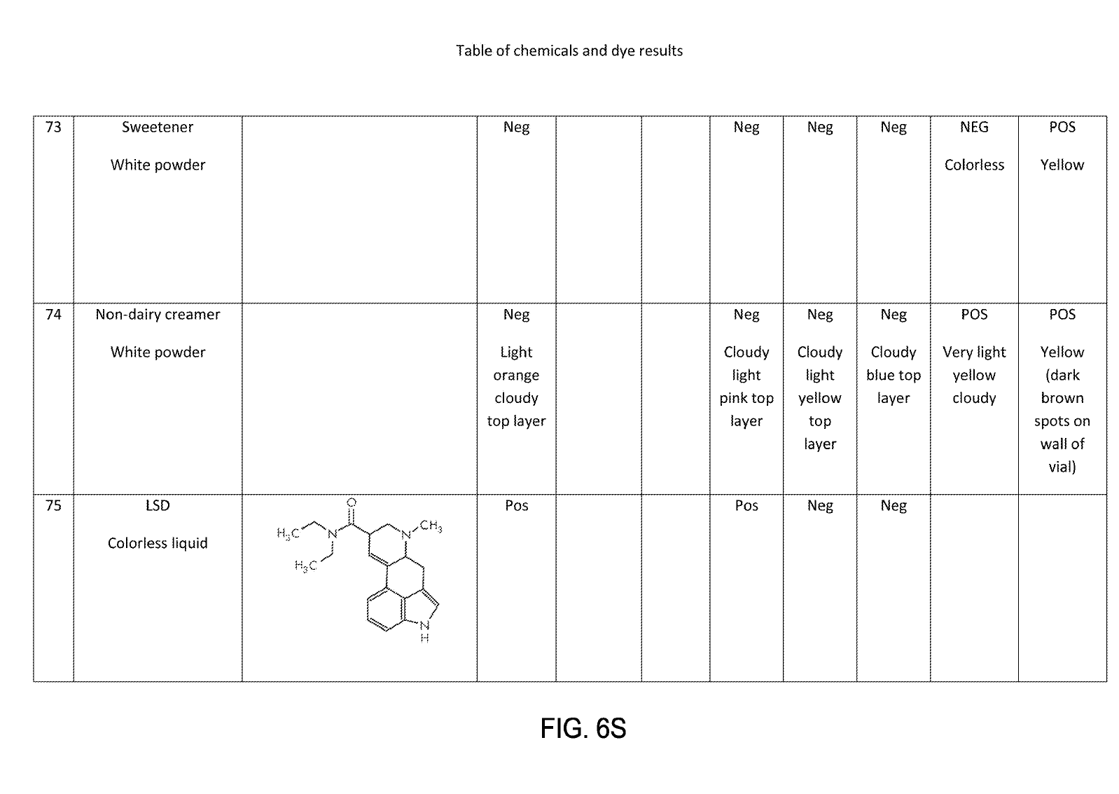

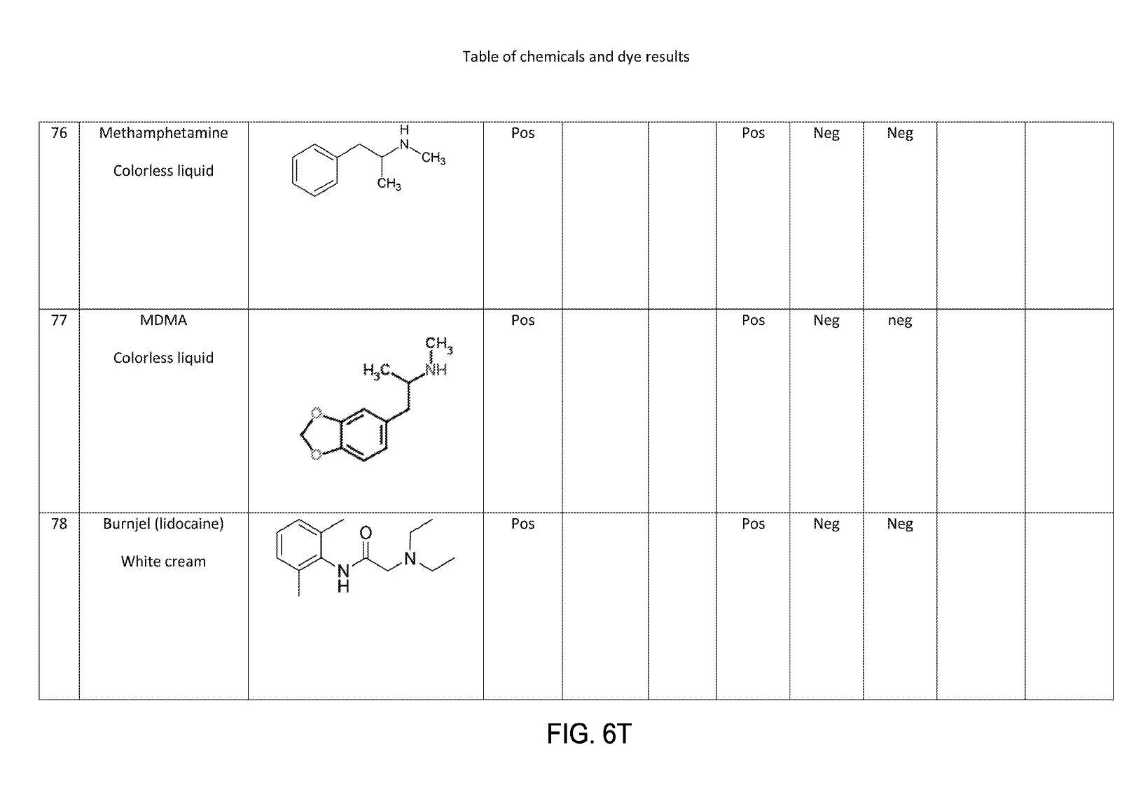

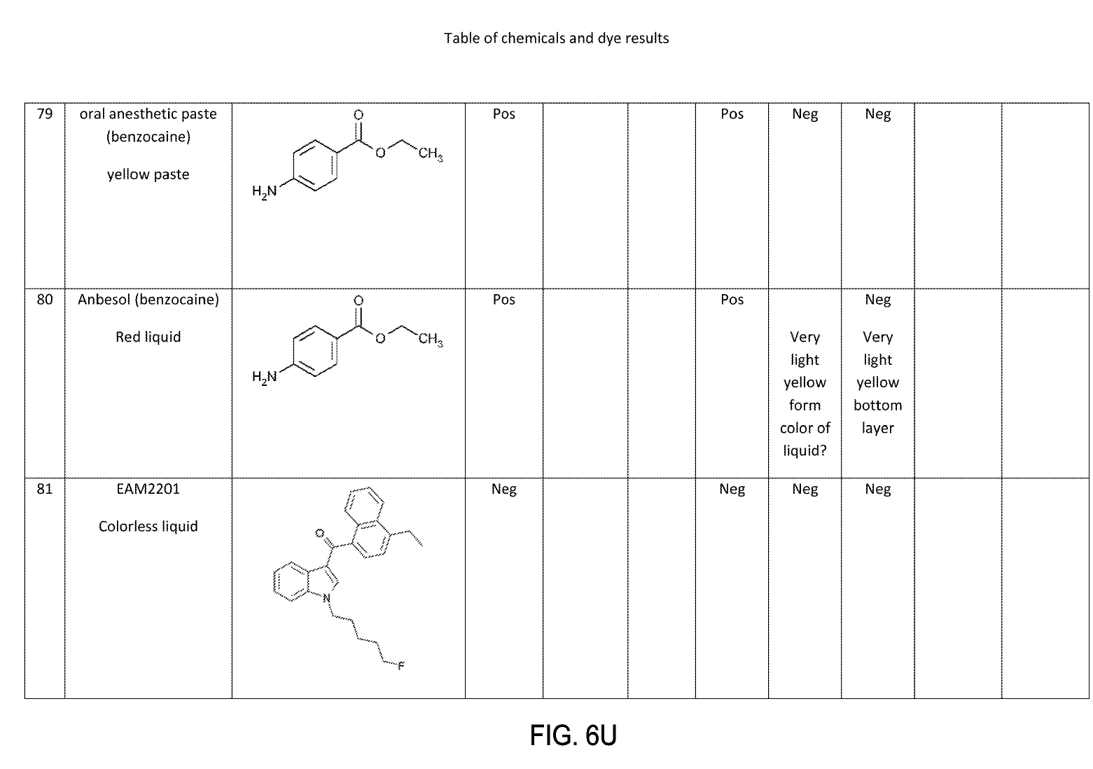

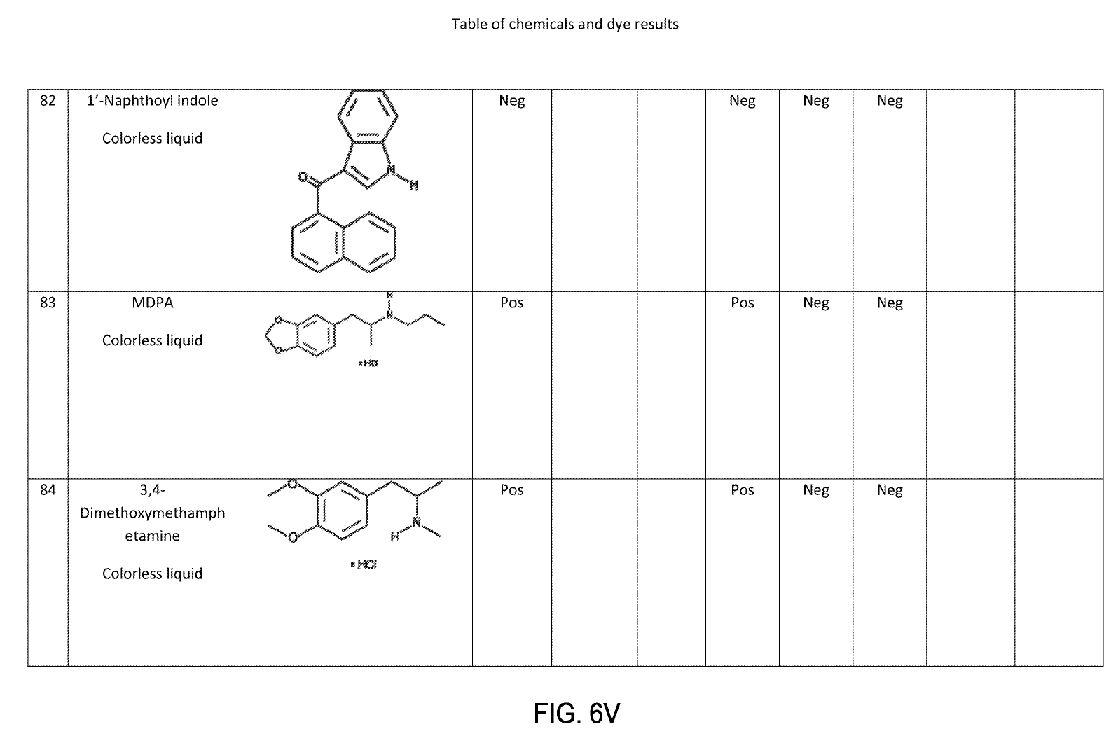

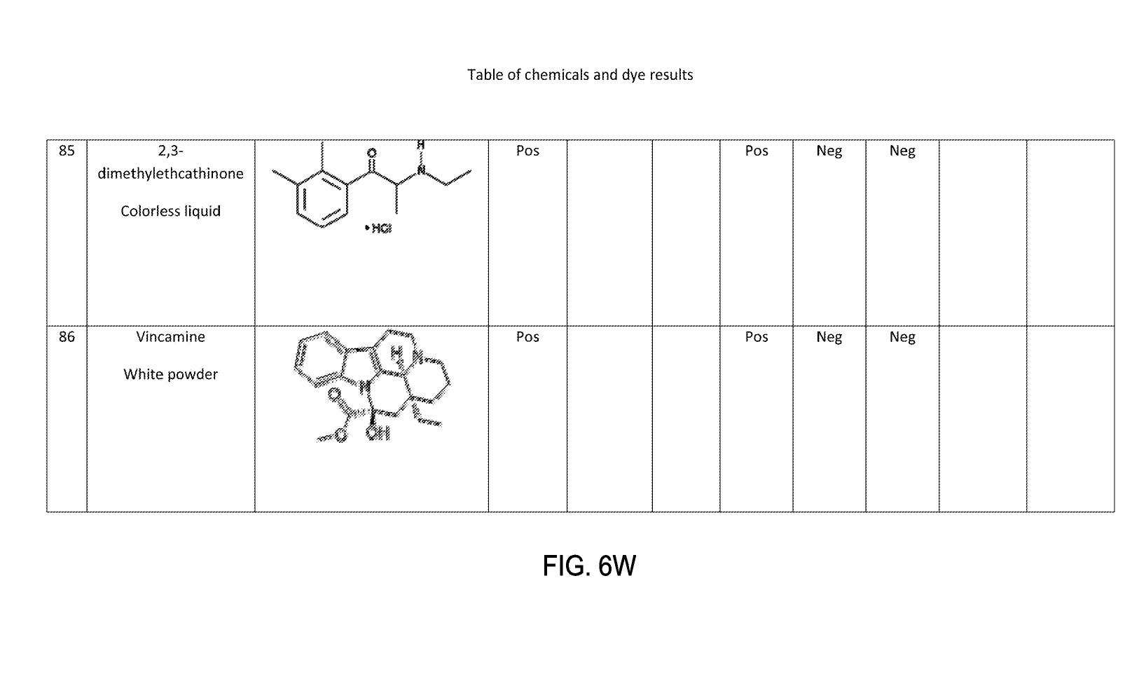

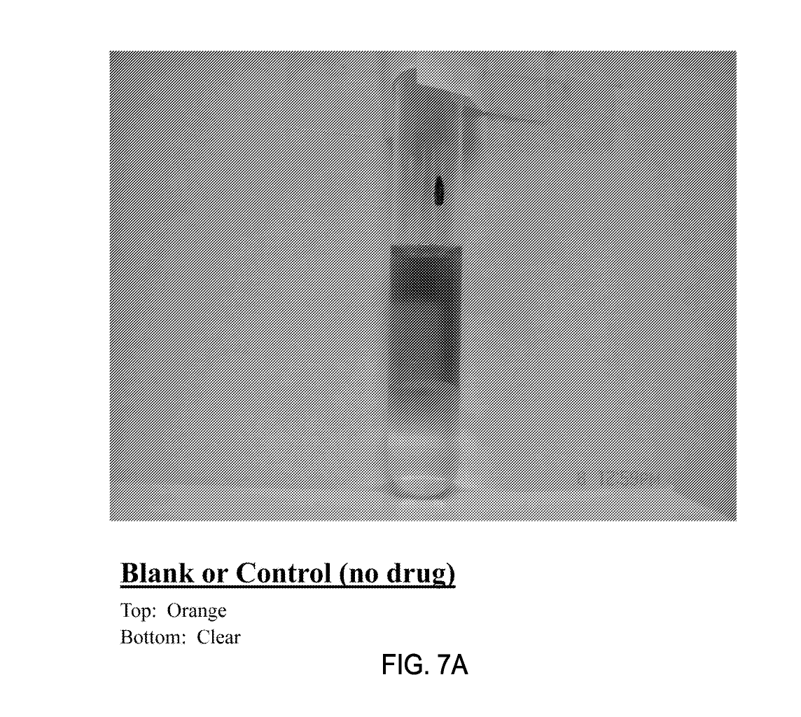

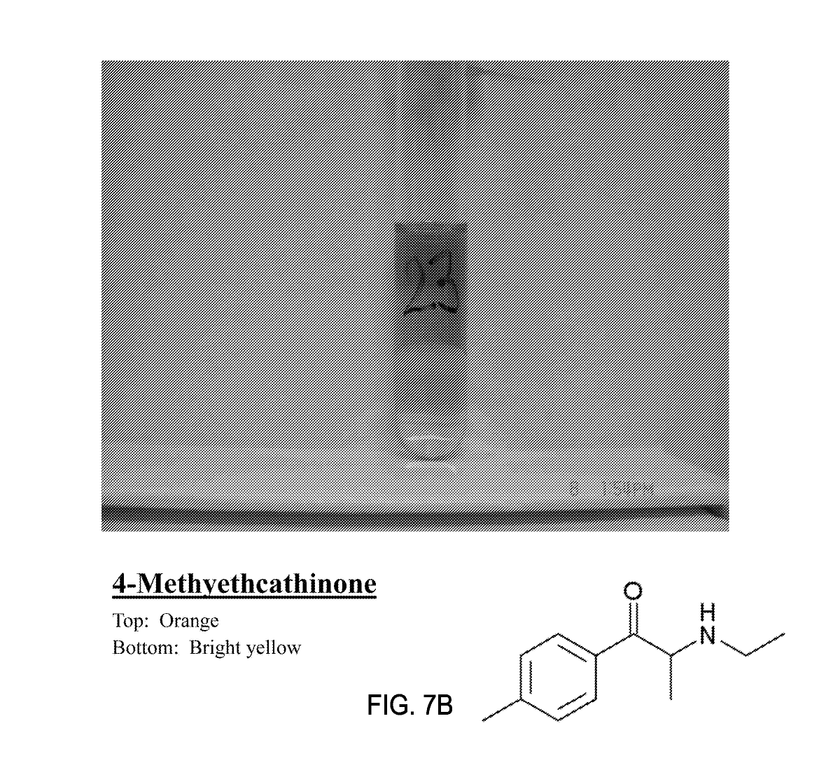

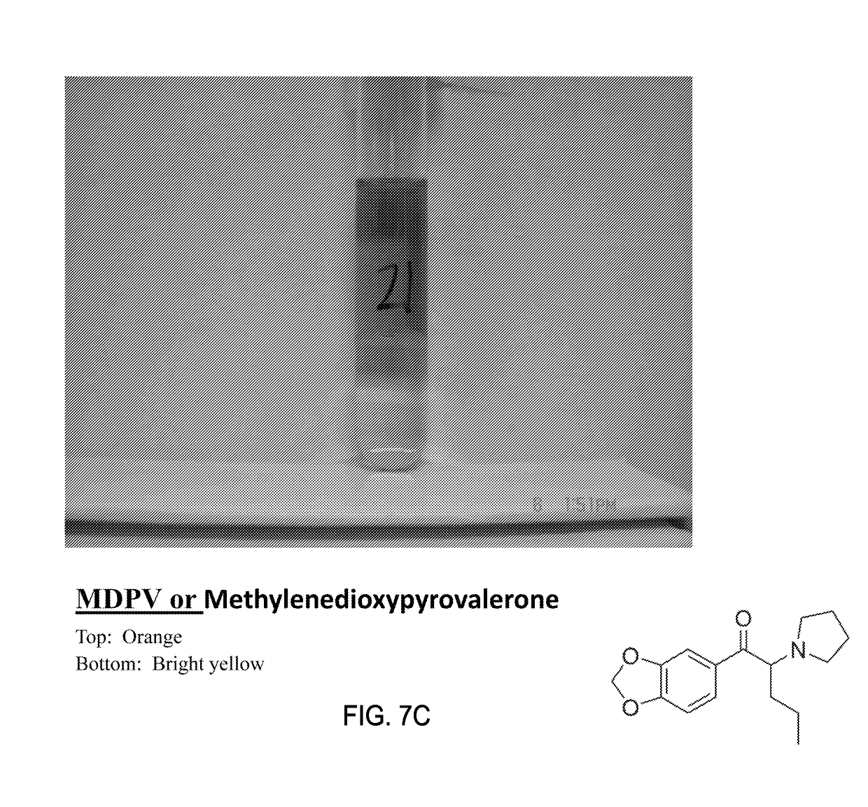

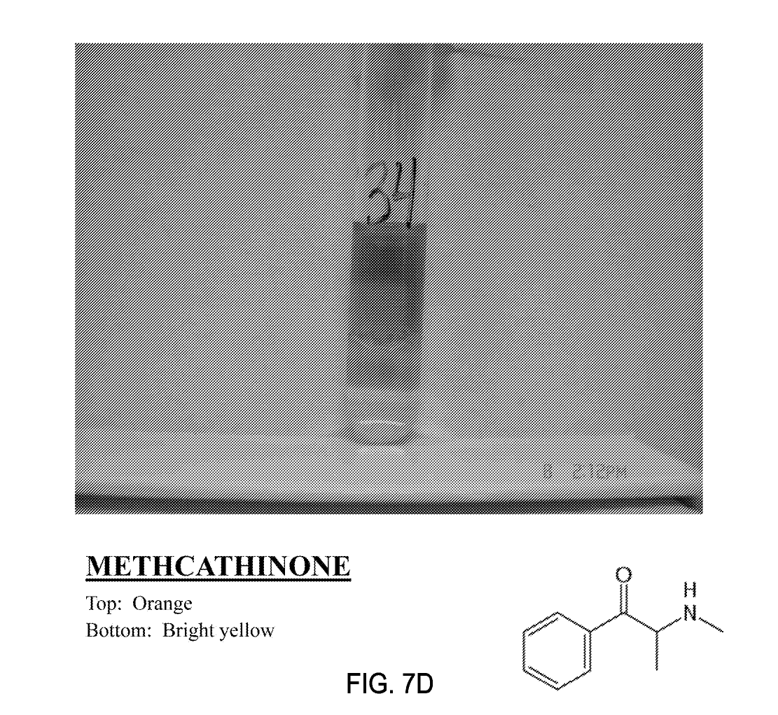

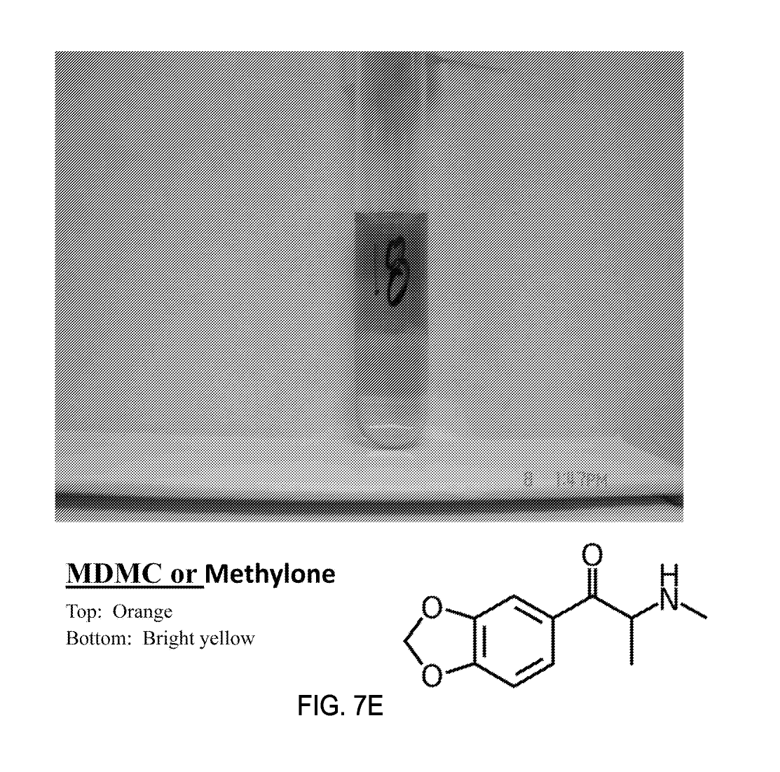

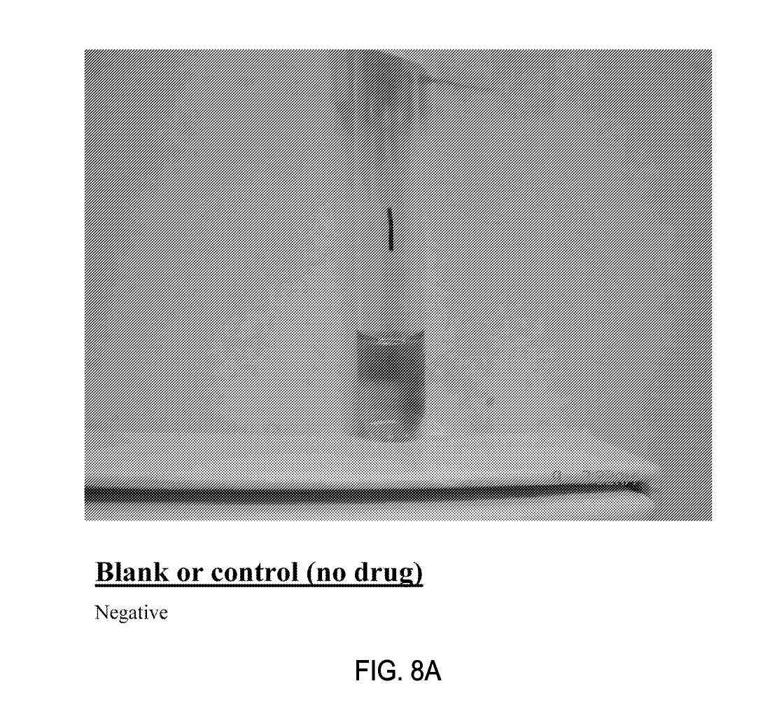

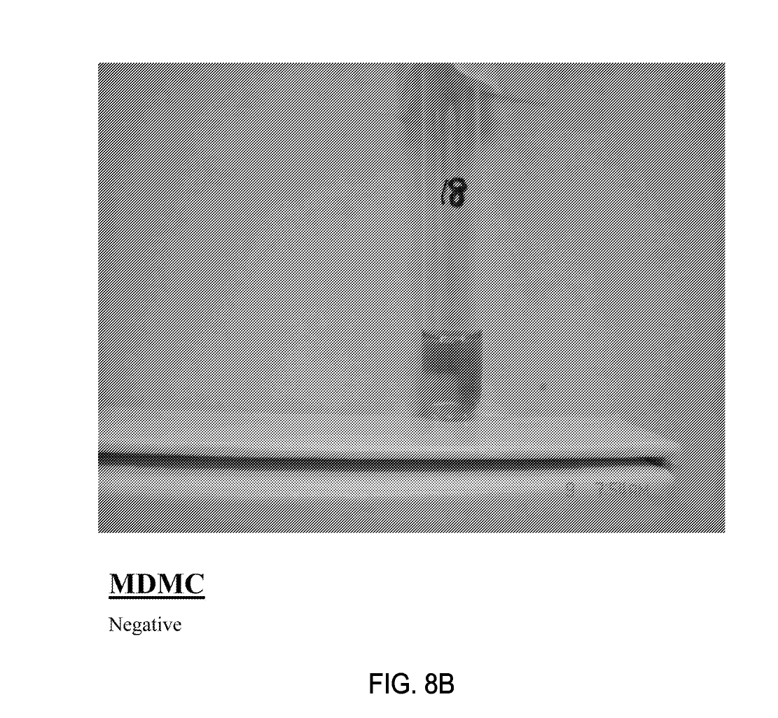

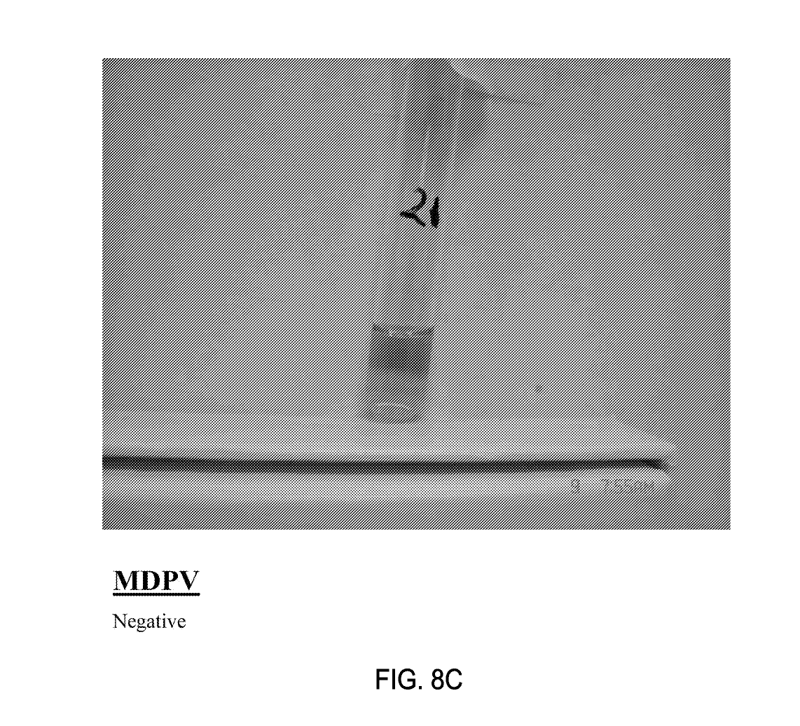

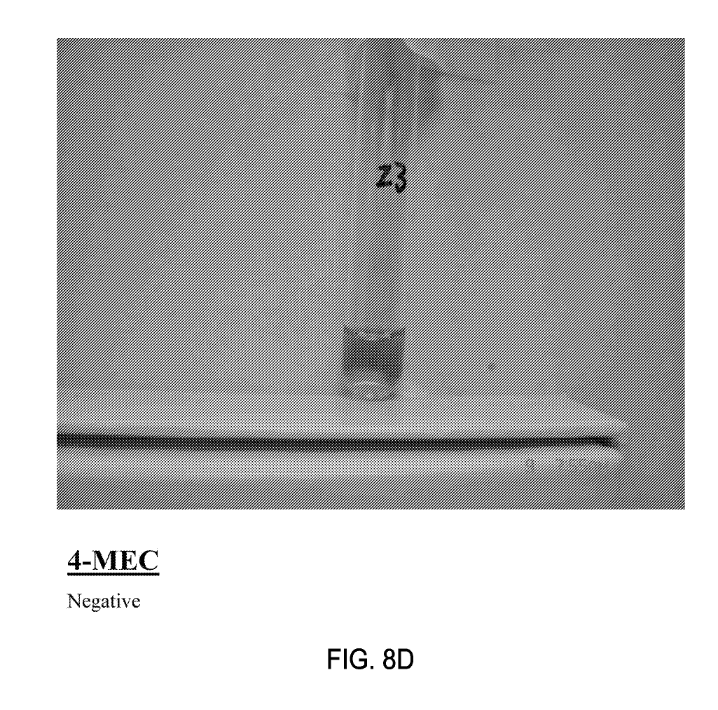

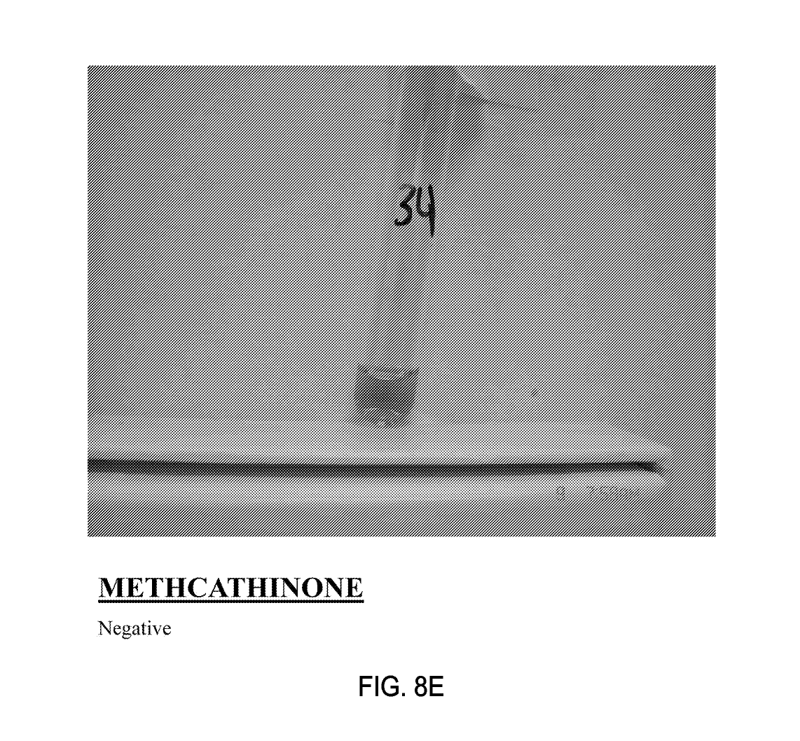

The detection is likely based on the ion-pairing ability and extractability of sulfonic acid, sultone or oxothian-2,2,dioxide containing dyes, such as methyl orange, xylenol orange, calmagite, metanil yellow, and 1-(2-hydroxyl-1-naphthylazo)-2-napthol-4-sulfonic acid zinc salt, with the controlled substance at an acidic pH, e.g., a pH of less than 7.0, including a pH of about 1, about 2, about 3, about 4, about 5, or about 6. In one embodiment, the assay includes placing about 10 milligrams of a suspected controlled substance (which may be present with other substances) in an ampoule (receptacle) containing about 1 mL of 0.1% dye, about 1 mL of pH 4 phosphate buffer, and about 1 mL of chloroform, although other volumes, other dye concentrations, other buffers and other organic solvents may be employed. After stirring vigorously, the bottom layer of the liquid in the ampoule will turn yellow for methyl orange dye and red for the 1-(2-hydroxyl-1-naphthylazo)-2-naphthol-4-sulfonic acid zinc salt if the controlled substance is present (see FIGS. 1 and 2).

Initially, cetirizine, an anti-histamine, was tested with methyl orange. Subsequently, a color change was detected with the assay when scheduled compounds, e.g., BZP, TFMPP, methamphetamine, or Ecstasy (MDMA), were tested. In one embodiment, a powder sample is added to a test kit and agitated to mix the reagents with the sample, e.g., for a few seconds. In the presence of compounds such as benzylpiperazine, MDMA, 2,5-dimethoxy-4-methylamphetamine (DOM), or a mixture of synthetic cannabinoids compounds, the test solution changed color from colorless to yellow. Furthermore, the intensity of the color correlated to the amounts of the drug that were added.

The reagents and compositions of the invention readily detected the presence of heroin, MDMA, methamphetamine, LSD, JWH018, synthetic cathinone, and cocaine.

One advantage of the present assay in the field is that it is not specific to the scheduled compounds and it is superior in identifying azabicyclo, morphinan, amine and indoline functional groups, which are commonly present in the schedule compounds. Existing color test kits (such as those in Table 1) are very specific to the type of scheduled compounds. However, this creates inconvenience for law enforcement officers in analyzing an unknown powder in the field as they have to test the same powder in different kits to identify the compounds. In contrast, the test kit described herein can detect various scheduled compounds in one test kit, which allows officers to screen a bag of probable drug materials in one test.

In one embodiment, the assay of the invention detects a piperazine, indoline, indole, azabicyclo, morphinan, or amine containing compound even when that compound is in the presence of other materials, including other scheduled compounds and plant materials, which are commonly found in the seized samples. Thus, Dragendorff's reagent and dye reagents disclosed herein useful in one embodiment have little if any reactivity with common substances, pharmaceutical compounds, and pesticides, such as those shown in Table 2 below.

TABLE 2
Substances that may be used as diluents.

| | | | |
|---|---|---|---|
| Acetaminophan | Aspirin | Baking Soda | Benzocaine* |
| Boric Acid | Caffeine | Chlorphenir- | Dextrome- |
| | | amine* | thorphan* |
| Dimethylsulfone | Diphenhydramine | Ephedrine* | Lidocaine* |
| Inositol | Mannitol | Ibuprofen | Procaine* |
| Quinine* | Tea extract | Benzphetamine* | Methysergide* |
| 1′-napthol indole | Parmesan Cheese | Sweetener | Powdered Milk |
| Pseudoephedrine* | Tetracaine* | Sucrose | Glucose |

*Indicates false positive result with methyl orange test

In one embodiment, the assay detects the presence of synthetic canninoids, e.g., those in Table 3.

TABLE 3
Synthetic cannabinoids
Controlled substances/Remarks
‘Spice’ products classified as medicinal preparations

CP-47
497-C6/C7/C8/C9
JWH-018
HU-210
JWH-015
JWH-019
JWH-073
JWH-250
JWH-398
JWH-200
JWH-018m
JWH-081
JWH-122
JWH-098
JWH-149
JWH-166
JWH-175
JWH-176
JWH-184
JWH-185
JWH-192
JWH-193
JWH-194
JWH-195
JWH-196
JWH-197
JWH-198
JWH-199
Leonotis Leonurus
Nymphacea caerulea
JWH-398

The assay may be employed to detect common scheduled compounds in four classes of compounds, e.g., narcotics, depressants, stimulants, hallucinogens, cannabinoids (both naturally occurring and synthetic cannabinoids), and cathinones (both naturally occurring and synthetic cathinones), e.g., such as 4-methyl-N-methylcathinone (mephedrone), 3,4-methylenedioxy-N-methylcathinone (methylone), and 3,4-methylenedioxypyrovalerone (MDPV). Synthetic cathinones include but are not limited to mephedrone, methylone, MDPV, butylone, 4-fluoromethcathinone (4-FMC), 3-fluoromethcathinone (3-FMC), 4-methoxymethcathinone (methedrone), 4-methyl-N-ethylcathinone (4-MEC), ethylone, buphedrone, dimethylcathinone, diethylcathinone, and 3,4-methylenedioxy-α-pyrrolidinopentiophenone (MDPBP). “Bath salts” are made from methcathinone analogues, typically mephedone and MDPV. Table 4 lists the targeted controlled compounds from common scheduled compounds in the four classes, representing narcotics, depressants, stimulants, hallucinogens, and specific cannabinoids.

TABLE 4

| Class | Targeted compound |
|---|---|
| Narcotics | Opiates |
| | Heroin |
| | Hydrocodone |
| | Morphine |
| Depressants | Barbiturates |
| | Benzodiazepines |
| Stimulants | Cocaine |
| | Amphetamines |
| | MDA (3,4-methylenedioxy- |
| | amphetamine) |
| | MDMA (3,4-methylenedioxy- |
| | methamphetamine) |
| Hallucinogens | Psilocybin |
| | Lysergic acid diethylamide (or LSD) |
| | Phencyclidine |
| Cannabinoids | THC |
| | Synthetic cannabinoids |
| | JWH 018 |
| | AM 2201 |
| Cathinones | Cathinones |
| | Synthetic cathinones |

Dyes useful in the invention have at least one sulfonic acid, sultone or oxothian-2,2,dioxide functional group that may form ion-pairs with compounds including illicit drugs that contain amine (primary, secondary or tertiary), piperazine (primary, secondary or tertiary), indoline and optionally indole groups (which may be present in synthetic cannabinoids and psilocybins and may be converted to indoline groups) functional groups. A receptacle of the invention includes an aqueous layer having the dye at the top and an organic solvent, e.g., chloroform, layer at the bottom. The test sample to be added may be in powder or liquid form. In one embodiment, Dragendorff's reagent can be incorporated to detect synthetic cannabinoids and alkalkoids.

Sulfonic acid containing dyes which may be useful in the methods and kits include those in U.S. Pat. No. 4,560,765 and those described in Clanton et al., J. Acquired Immune Defic. Syndr., 5:771 (1992), the disclosures of which are incorporated by reference herein. For example, the following dyes may be useful in the assays described herein: suramin, sulfonic acid-containing azo compounds, 3′-azido-3′-dideoxycytidien (ddC), Chicago sky blue, Evan's blue, trypan blue, direct orange 15, direct blue 15, Erie fast blue, Ink blue, Direct red 75, Erie yellow, and oxathiin carboxyanilide (structures of some of those dyes, as well as methyl orange and xylenol orange are shown below).

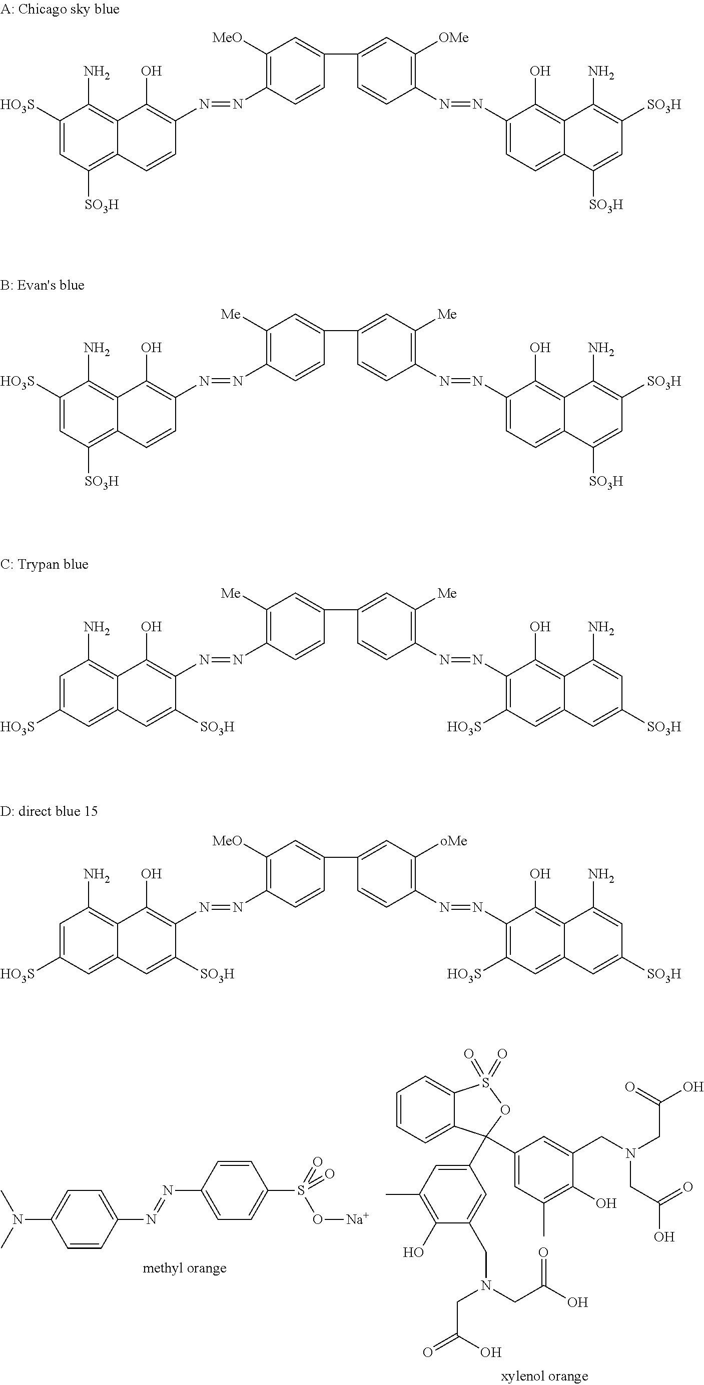

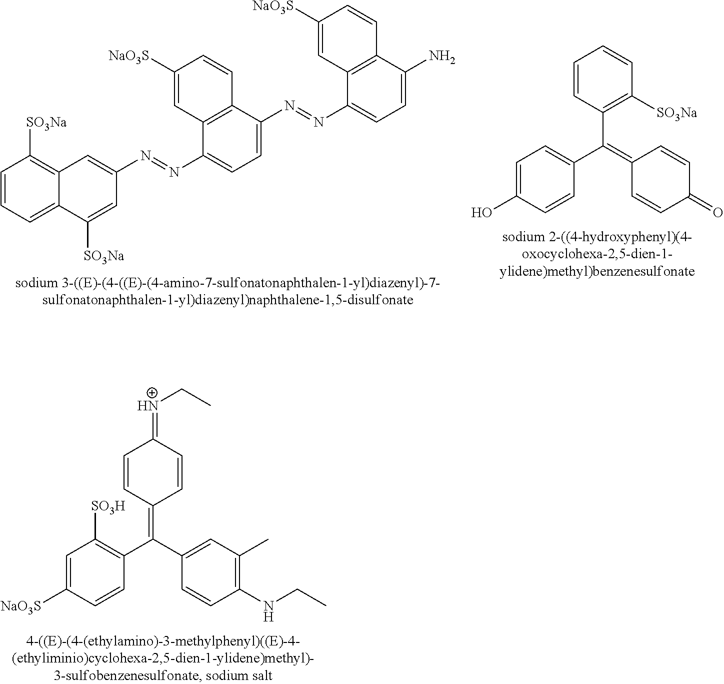

The use of color test kits is the most common method for the presumptive identification of drugs in the field because they are quick, easy to use, and cost effective, thus they are an ideal method for use in a Field Investigative Drug Officer (FIDO) program. Such color test kits provide clear, unambiguous color to indicate a positive or negative result, sufficient specificity to minimize false positive or false negative interpretation, adequate sensitivity to allow the detection of drugs at concentrations commonly encountered in street samples, accurate results for drugs mixed with a variety of adulterants, and reproducible results.

To determine if a particular dye, buffer and organic solvent combination is useful to detect and/or quantify a particular compound in a sample, the following test may be conducted. One (1) milligram to ten (10) milligram portions of each sample are weighed. Sensitivity is detected with samples tested in duplicate. Specificity is detected with samples tested in duplicate. Reproducibility is detected with ten (10) replicates. A single test of unknown samples is conducted. For example, a one (1), three (3) or ten (10) milligram portion is placed in each test pouch. After about sixty (60) seconds, the hue, value, and/or chroma of the final color for each sample may be recorded, referencing The Munsell Book of Color.

For example, two types of ampoules were prepared. A methyl orange ampoule was prepared using methyl orange dye and chloroform while a Dragendorff ampoule was prepared using bismuth nitrate and potassium iodide.

For powder samples, approximately 15 mg of the unknown powder is placed into the ampoule and shaken for 30 seconds. The color change is observed for methyl orange test, or precipitate formation is observed for the Dragendorff test.

For liquid samples, approximately 1 mL of liquid is poured into the ampoule and shaken for 30 seconds. The color change is observed for methyl orange test, or precipitate formation is observed for the Dragendorff test.

For leaf samples, approximately 0.5 g of leaves are placed into the ampoule and shaken for 30 seconds. The color change is observed for methyl orange test, or precipitate formation is observed for the Dragendorff test.

The results for compounds containing an amine functional group are shown in Table 5.

| | | MO | D | Marquis |
|---|---|---|---|---|
| Class | Target compound | Test | Test | Test |
| Narcotics | Heroin | + | + | Purplish |
| | | | | red |
| | Hydrocodone | + | + | |
| | Oxycodone | + | − | violet |
| | Morphine | + | + | purple |
| Depresants | Secobarbital | − | − | |
| | methaqualone | − | + | no |
| | | | | reaction |
| | Phenobarbital | − | − | |
| Stimulants | Cocaine HCl | + | + | no |
| | | | | reaction |
| | Cocaine Base | + | + | no |
| | | | | reaction |
| | Amphetamine | + | − | reddish |
| | | | | orange |
| | Ephedrin | + | − | no |
| | | | | reaction |
| | Lisdexamfetamine | + | − | |
| | Meth | + | − | reddish |
| | | | | orange |
| | MDMA (3,4- | + | − | dark |
| | methylenedioxy- | | | purple |
| | methamphetamine) | | | |
| Hallucinogens | Psilocybin | − | − | Brown |
| | Lysergic acid | + | − | Black |
| | diethylamide | | | |
| | (or LSD) | | | |
| | Phencyclidine | − | − | no |
| | (or PCP) | | | reaction |
| Cannabinoids | THC | + | − | Orange |
| | JWH 018 | − | − | yellow |
| | AM 2201 | − | − | |
| Synthetic | Cathinone | + | − | no |
| Cathinones | | | | reaction |
| | MDPV | + | − | Yellow |
| | 4-methylethcathinone | + | − | yellow |
| | Methcathinone | + | − | no |
| | | | | reaction |
| | MDMC | + | − | yellow |
| | 4-fluoromethcathinone | r+ | − | no |
| | | | | reaction |
| | 2 methoxymethcathinone | r+ | − | no |
| | | | | reaction |
| | methedrone | r+ | − | yellow |
| | butylone | + | − | yellow |
| | 3-methylbuphendrone | + | − | no |
| | | | | reaction |
| | 3,4 dimethylethcathinone | + | − | no |
| | | | | reaction |
| | 3,4 methylendioxy-α- | + | + | yellow |
| | pyrrolidinopiophenone | | | |
| | 3-fluoromethcathinone | + | − | no |
| | | | | reaction |
| | 2,3 dimethyl- | o+ | − | yellow |
| | ethcathinone | | | |
| Steroids | methandienone | − | − | |
| | testosterone | − | − | |
| Others | carisoprodol | − | − | |
| | diazepam | − | + | |
| | alprazolam | − | − | |
| | mitragynine | + | − | |
| | lorazepam | − | − | |
| | γ-Hydroxybutyric acid | − | − | |

According to the results, all tested synthetic cathinones showed positive in methyl orange test (meaning color change in the chloroform layer), most of tested synthetic cathinones showed negative in a Dragendorff's test (no precipitate), but the third test, the Marquis test, showed mixed results. The synthetic cathinones produced either no reaction or a yellow color in Marquis test which may be explained by two different reactions with the different molecular structures of the synthetic cathinones.

In summary, the results show that methyl orange test is very effective in detecting synthetic cathinones (bath salts) and may be teamed with Dragendorff, an existing test and/or a chemical test which can be used to eliminate false positives in synthetic cathinones detection. Additional tests such as Liebermann, Chen-Kao (Chen), Simon, Duquenois-Levine, Scott, Mecke, iodoform test, 2,4-dinitrophenylhydrazine test, Schiff's reagent, and/or tests measuring the presence of amine groups and carbonyl groups (e.g., the iodoform test, 2,4-dinitrophenylhydrazine test, or Schiff's reagent may be employed to detect carbonyl groups), can be employed rather than the Marquis to assist in reducing the rate of false positives.

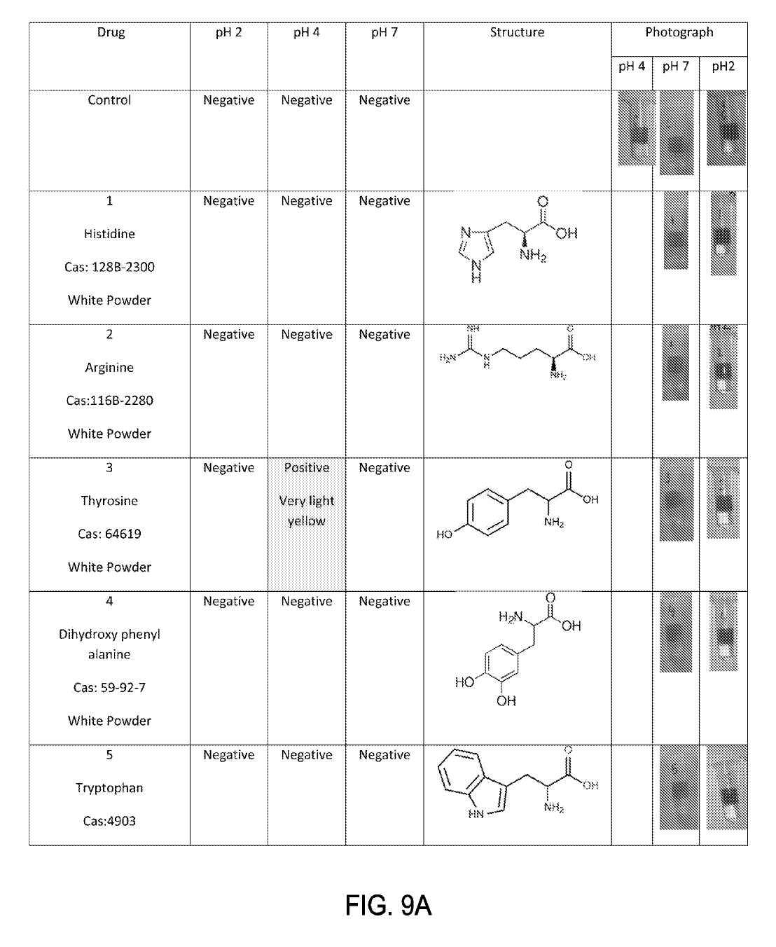

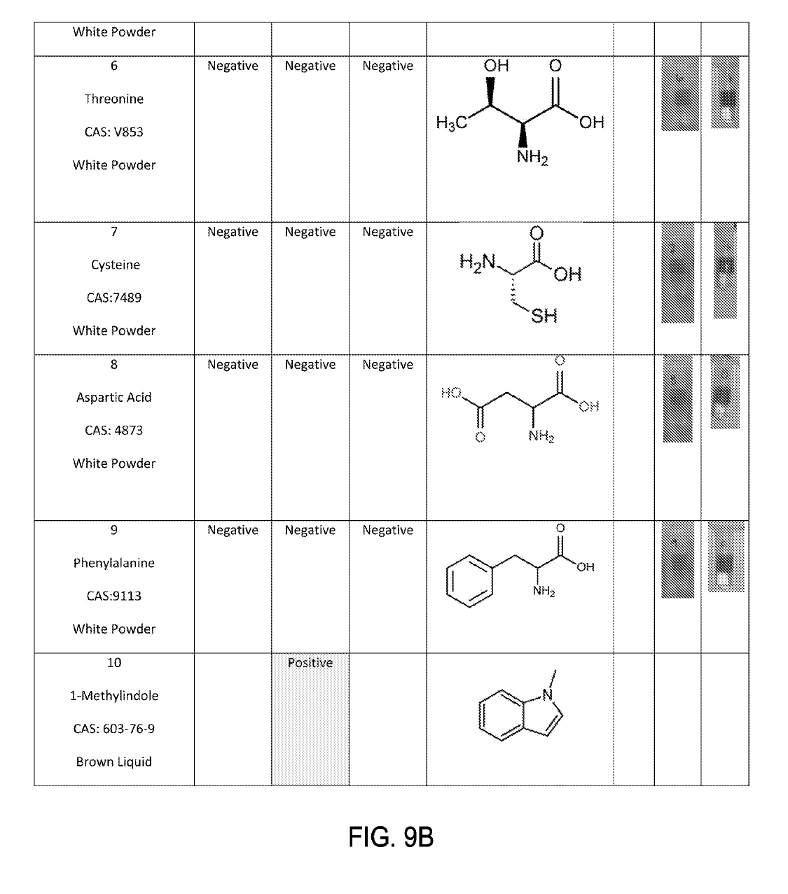

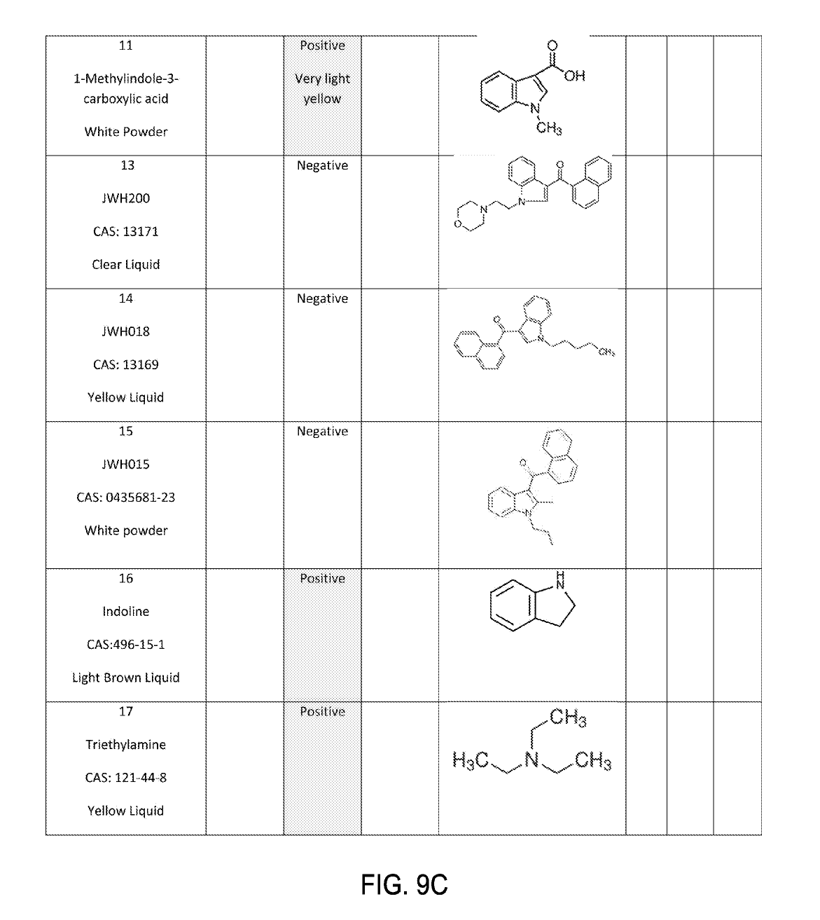

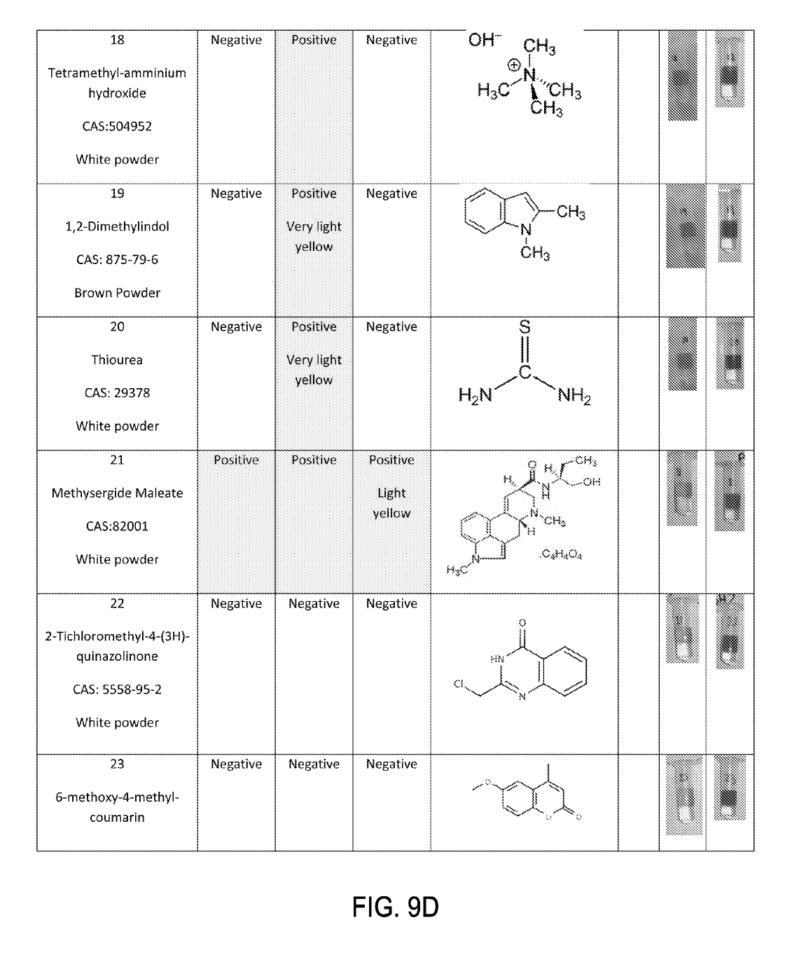

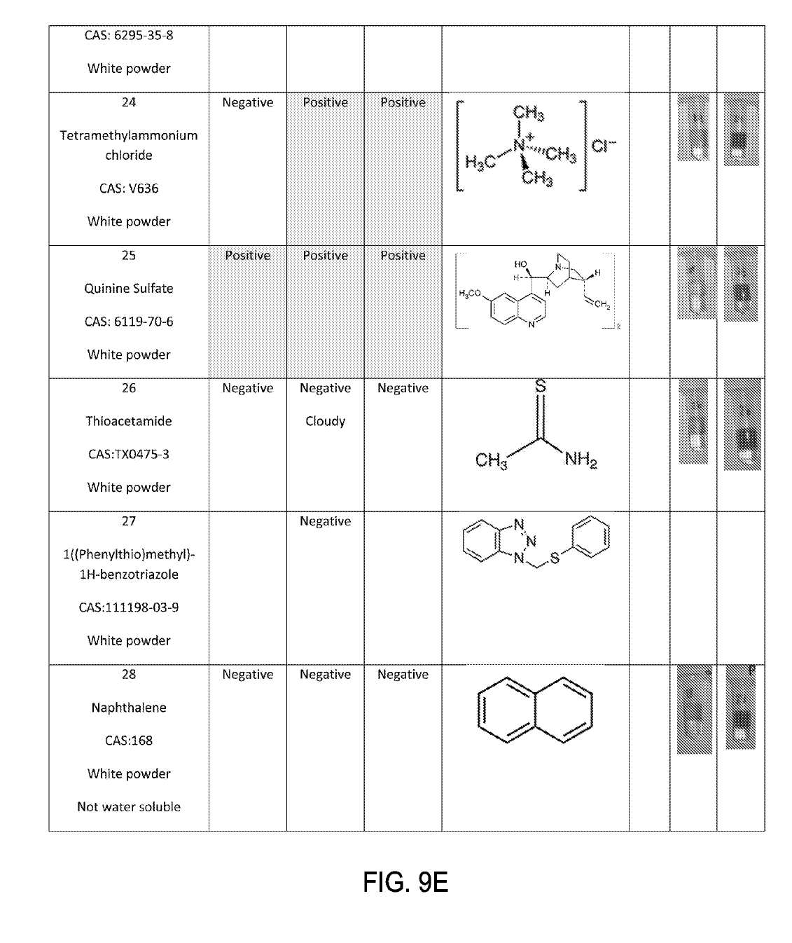

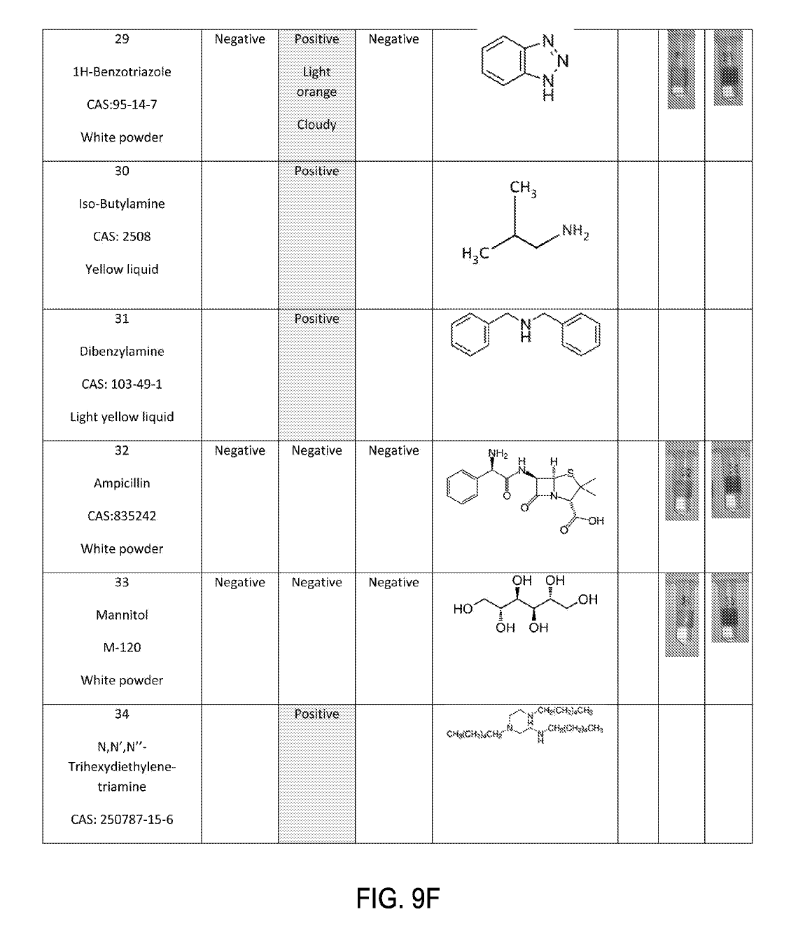

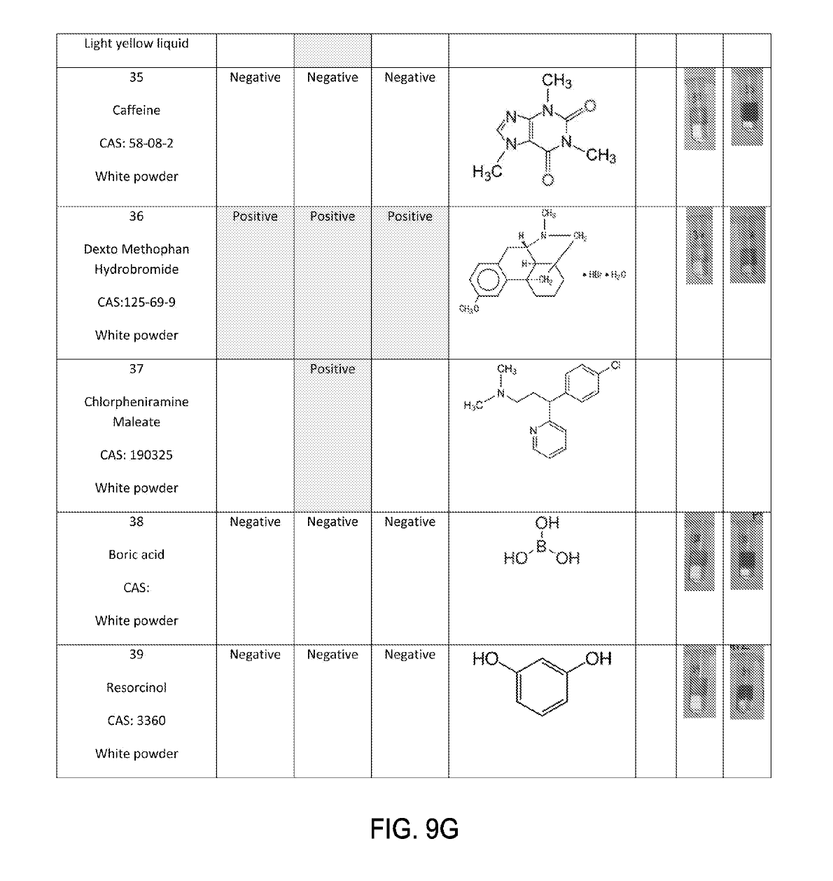

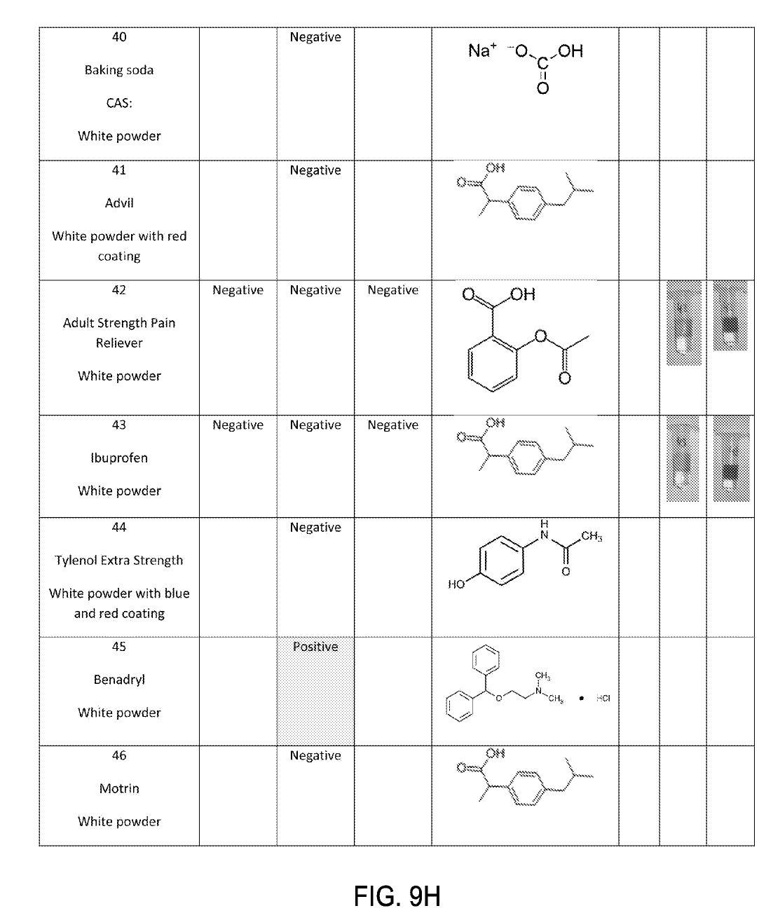

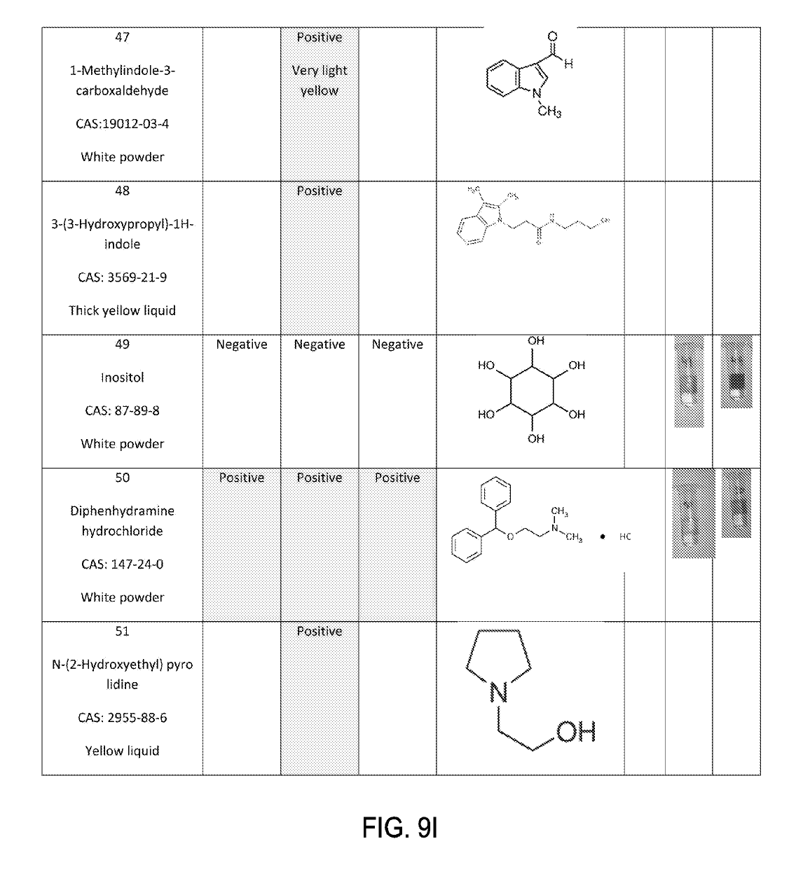

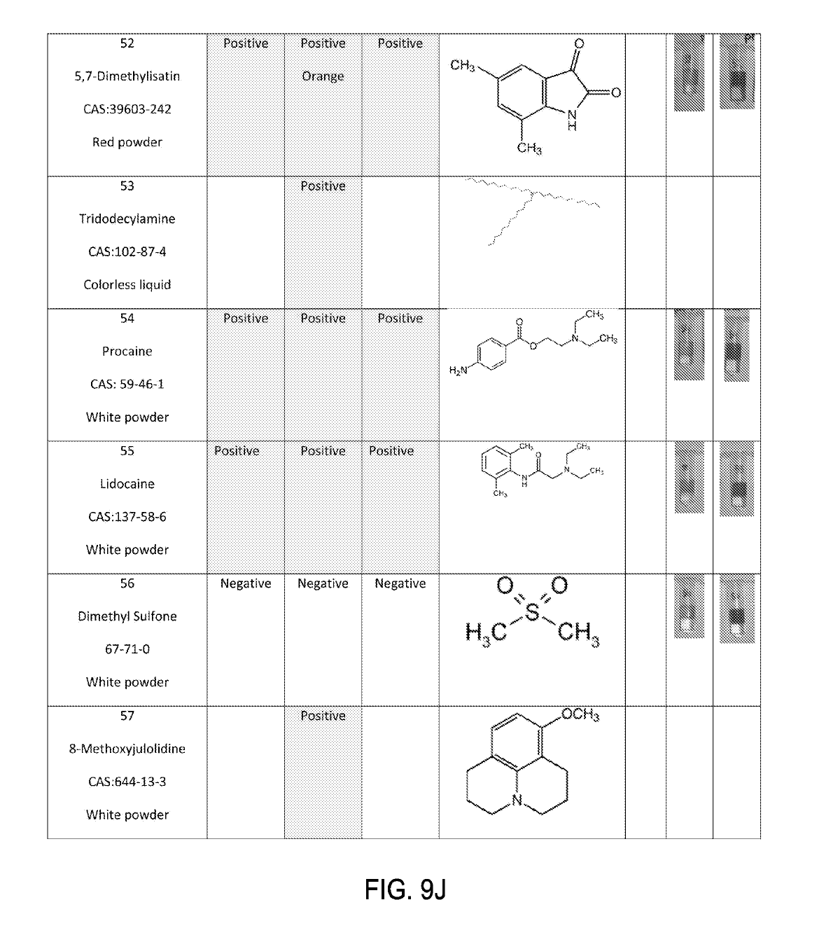

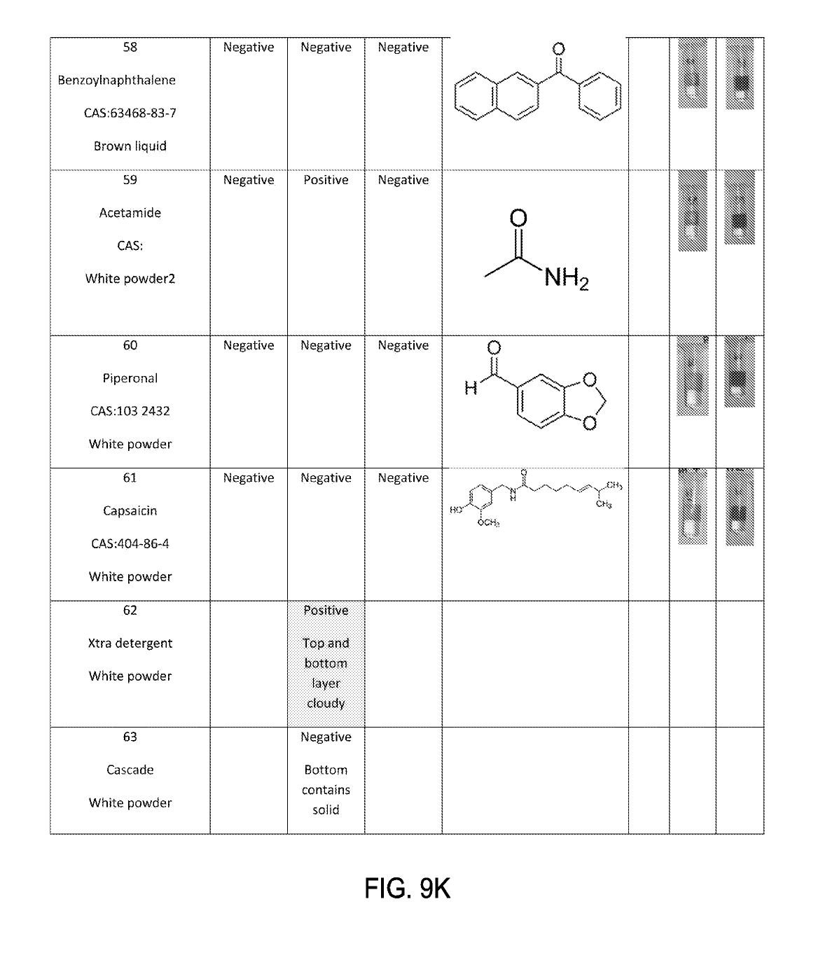

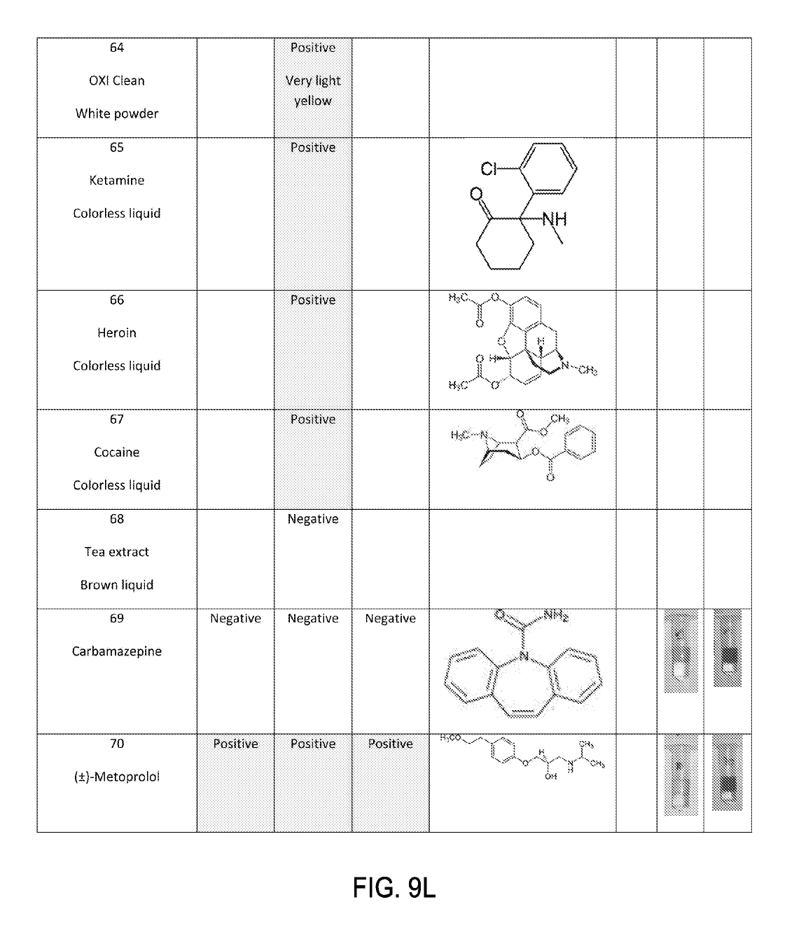

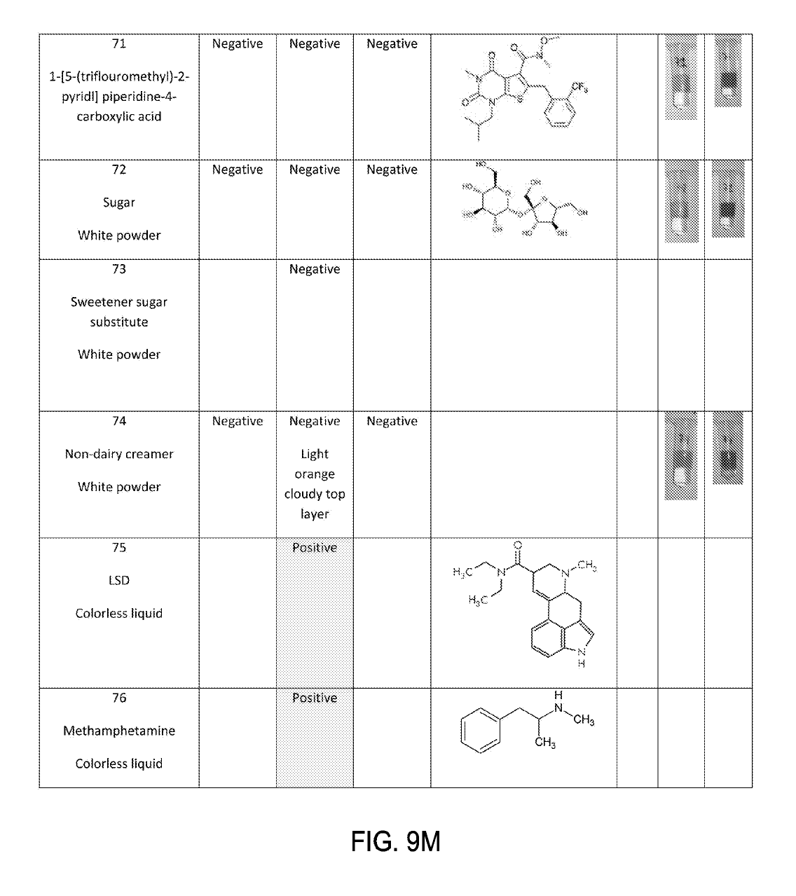

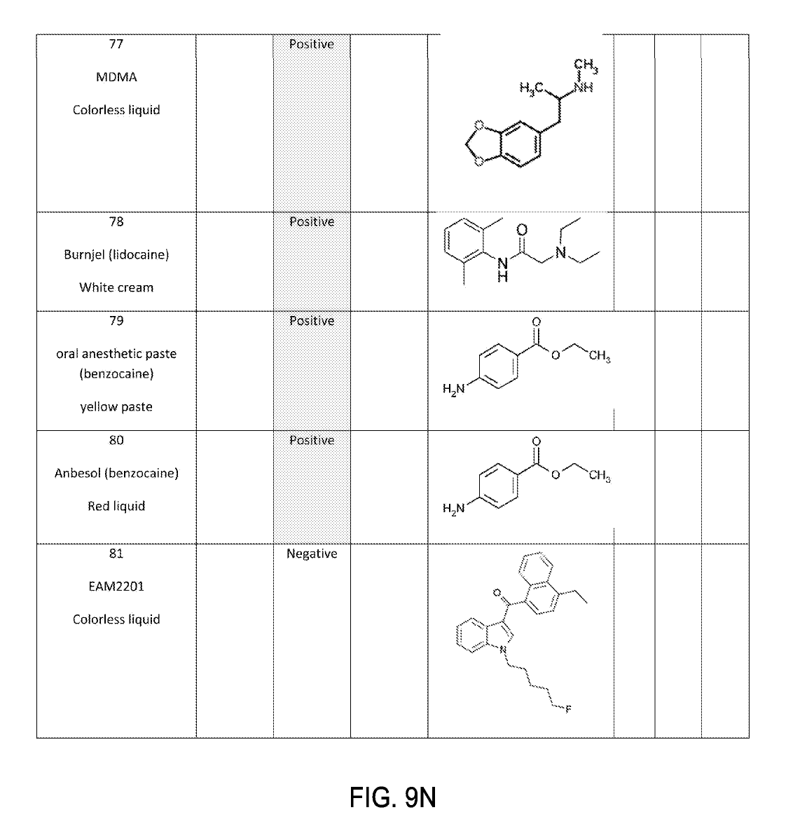

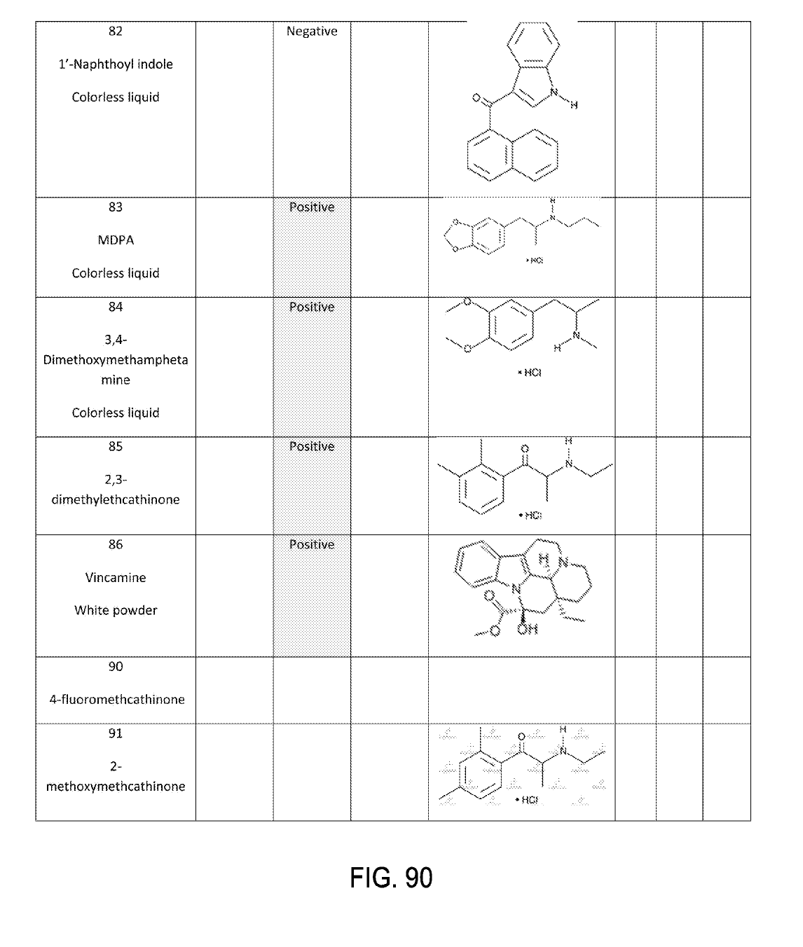

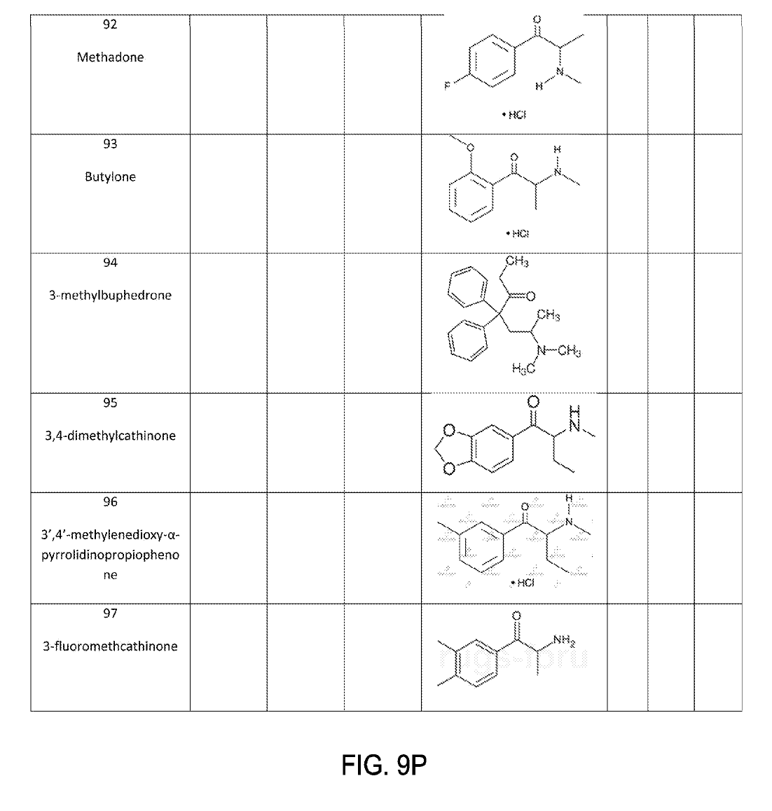

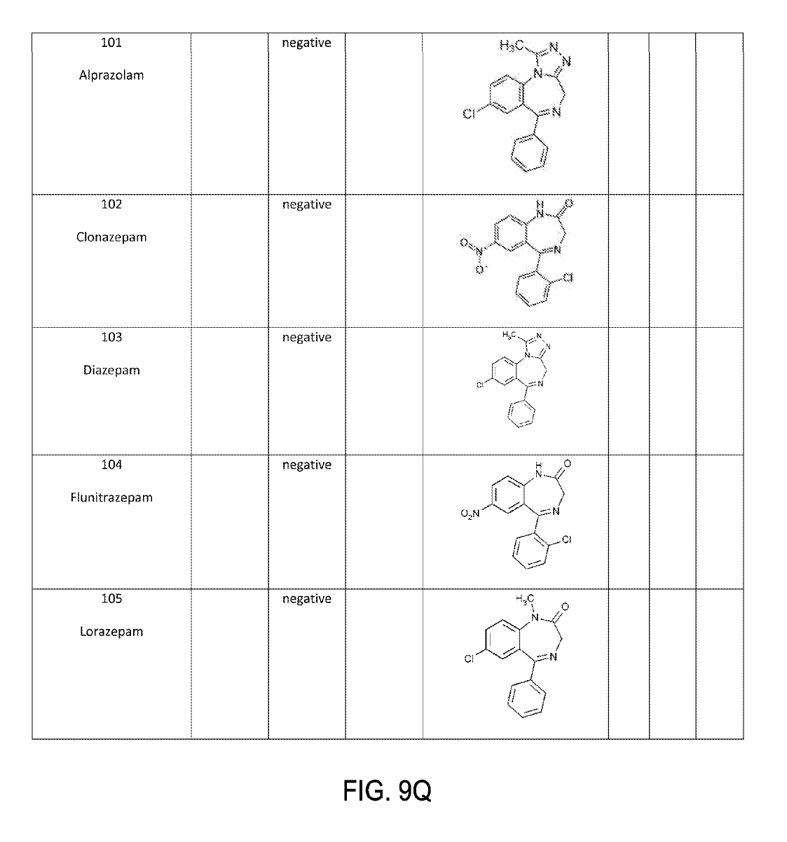

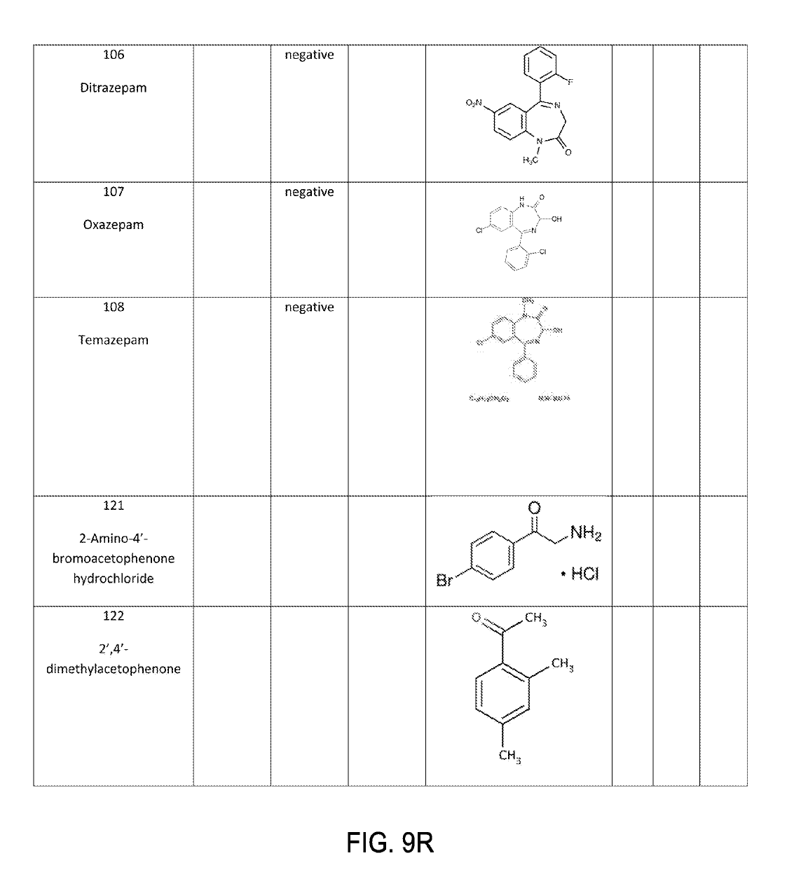

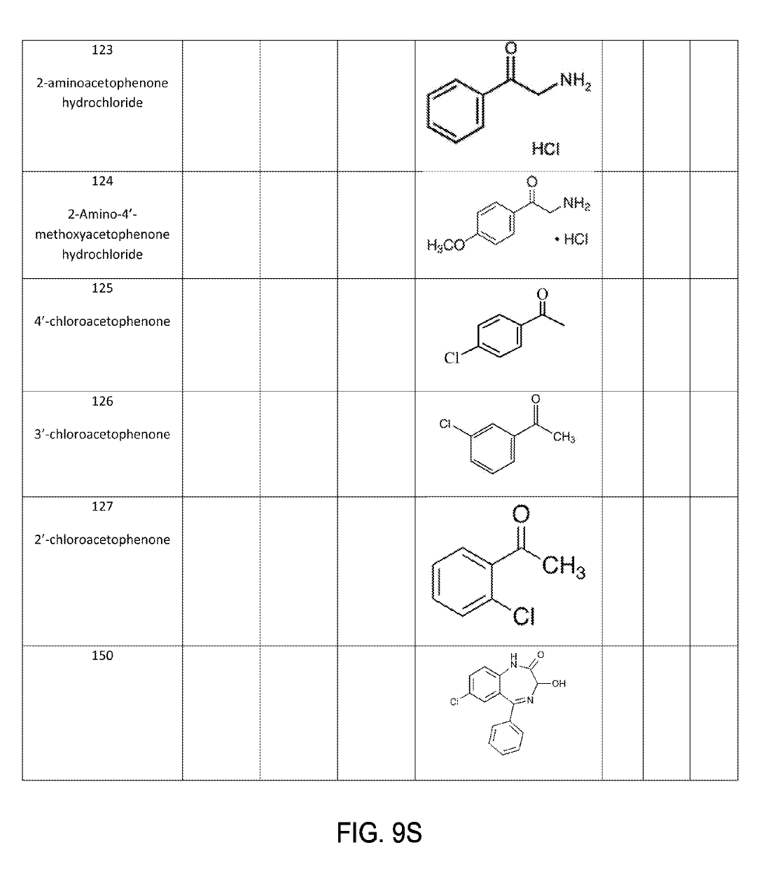

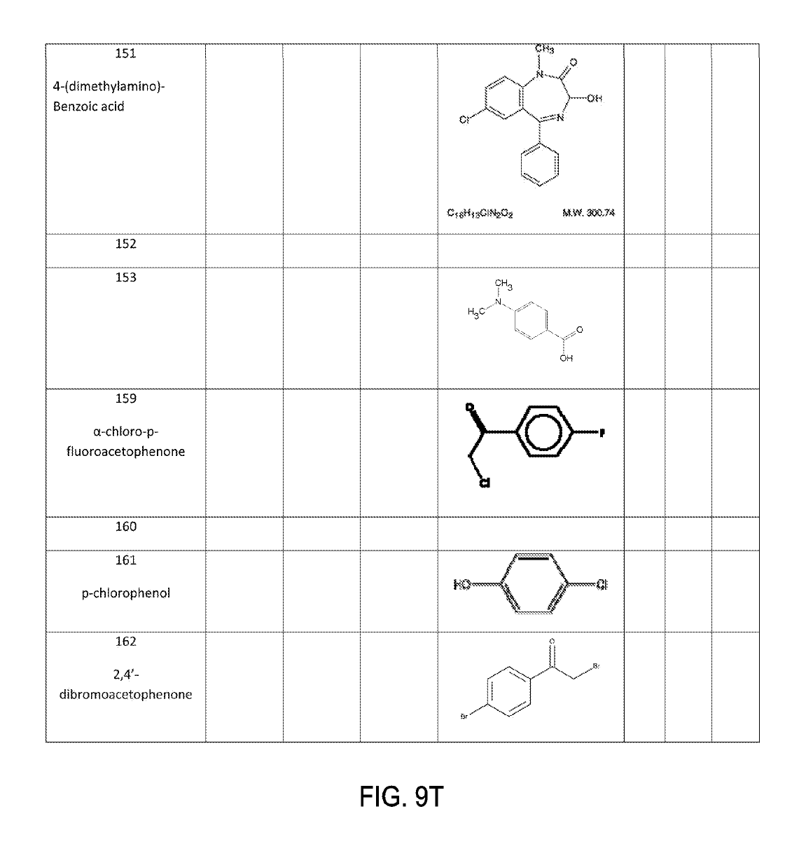

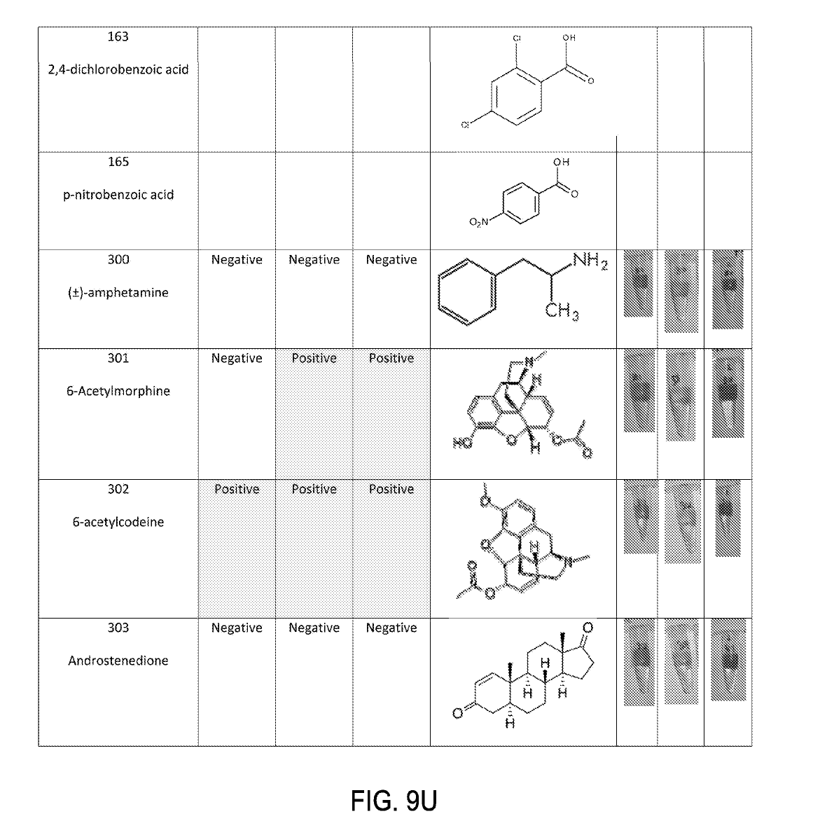

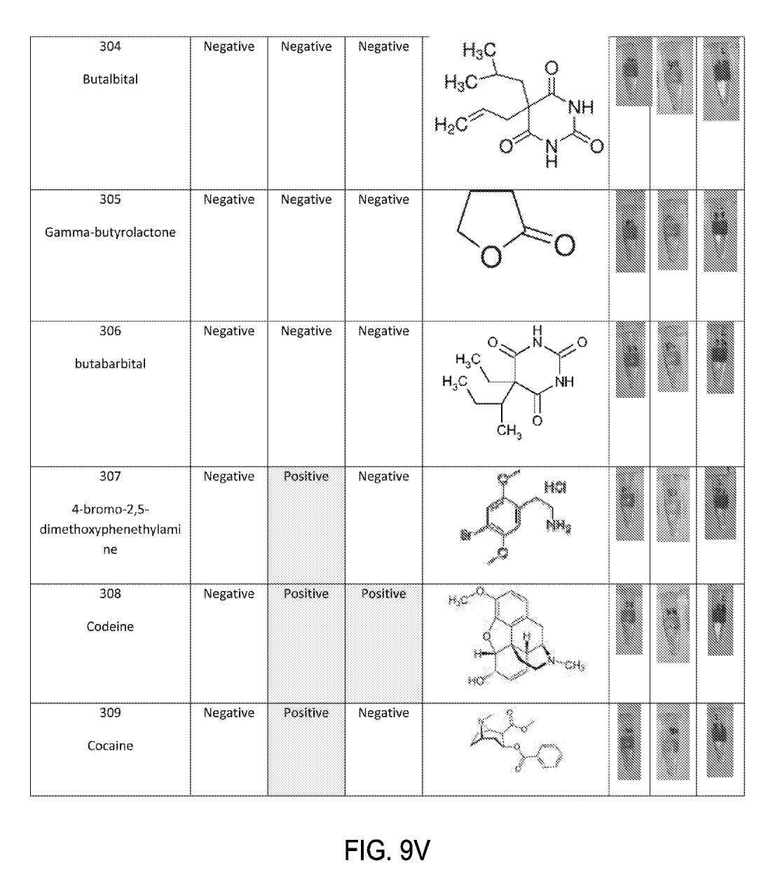

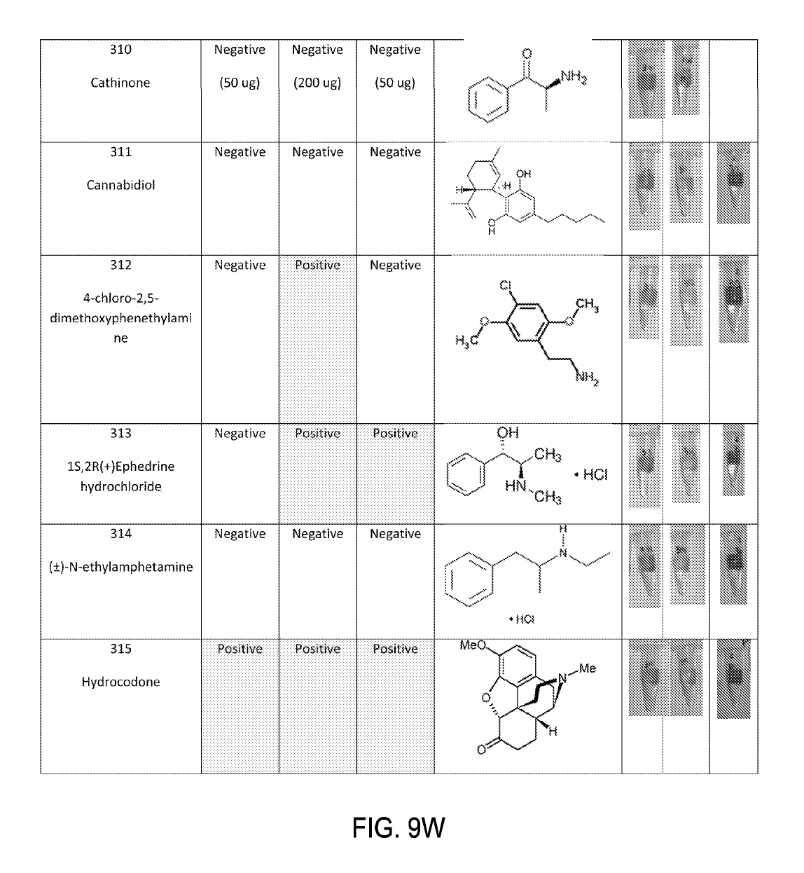

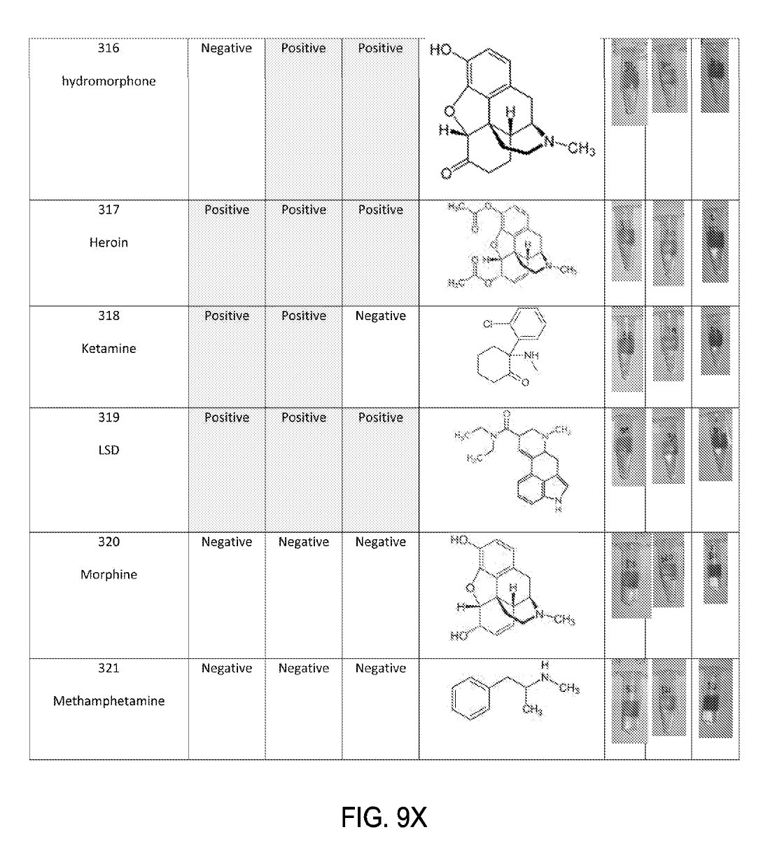

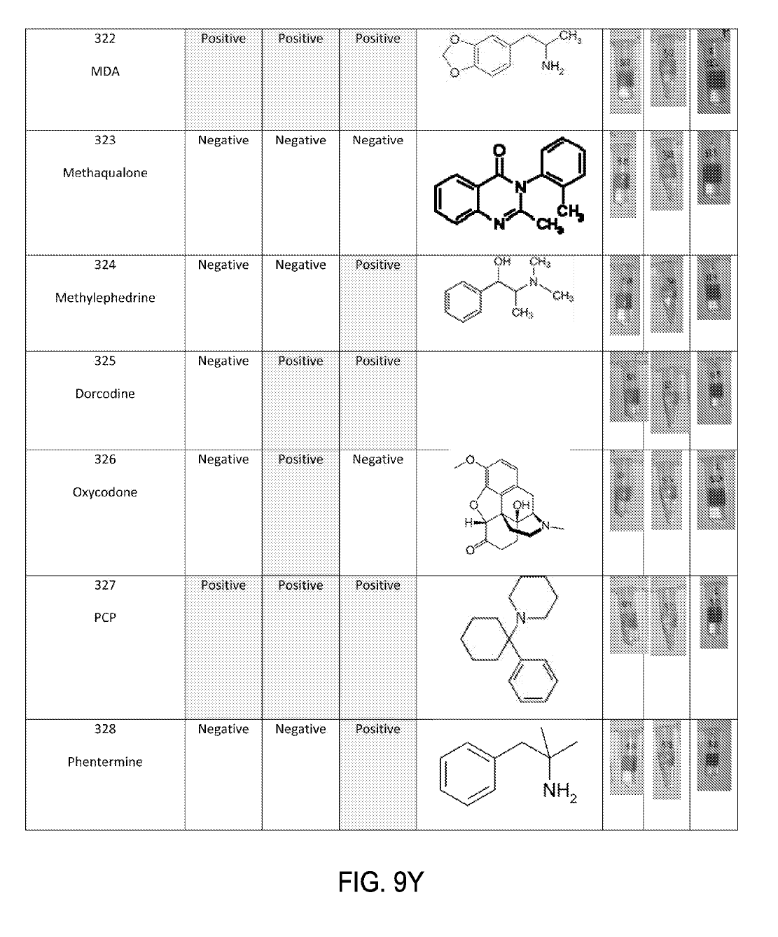

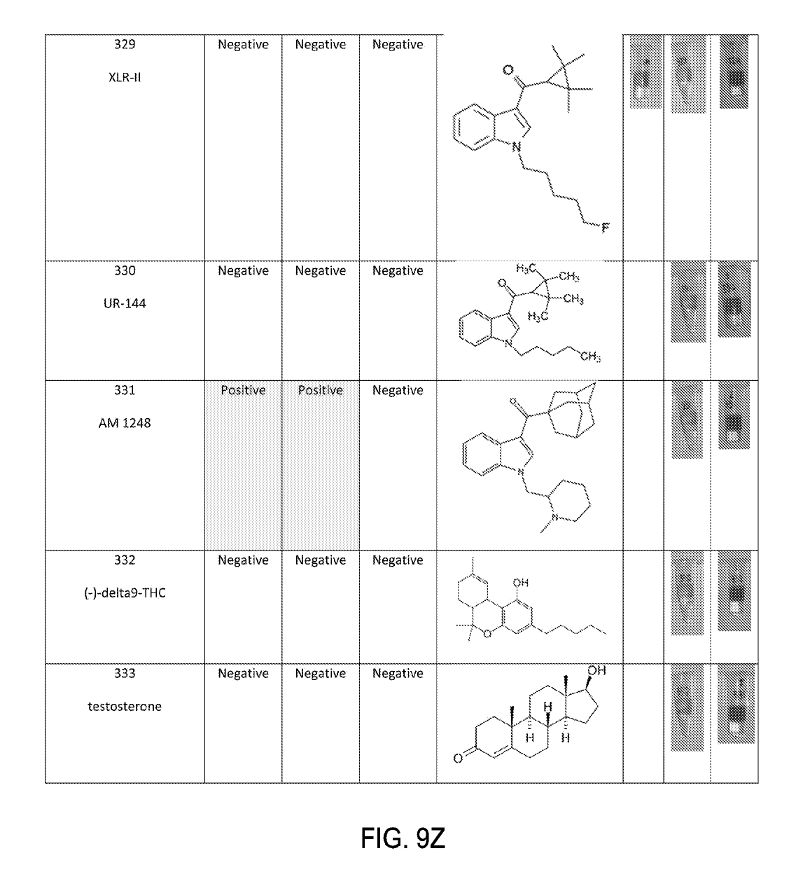

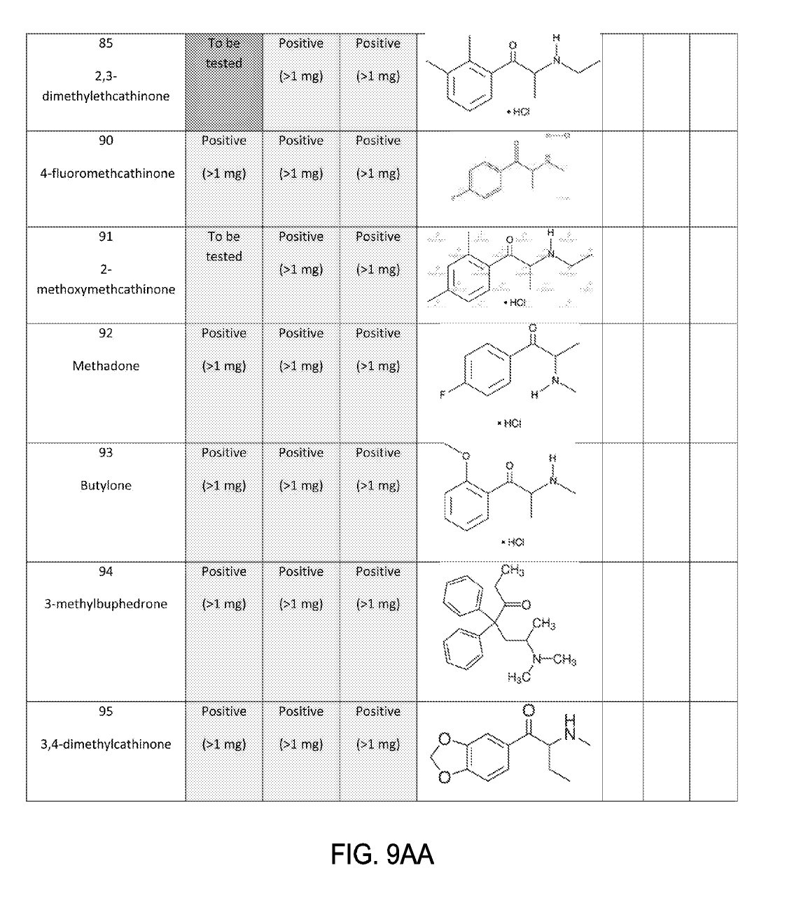

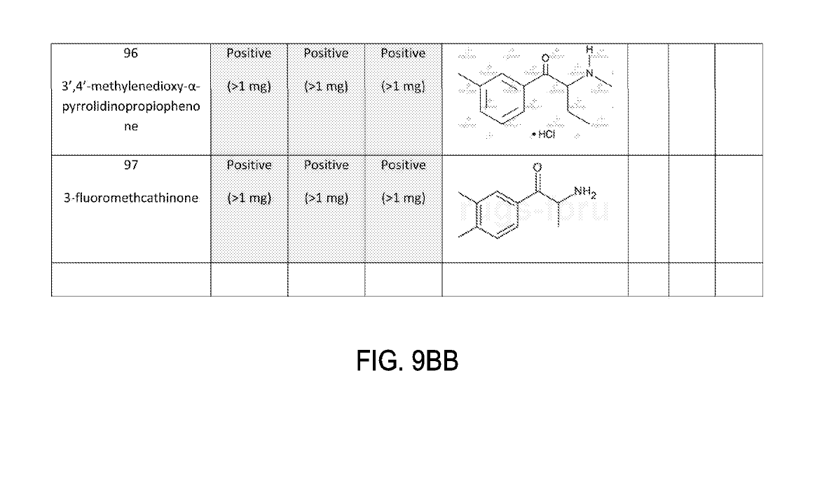

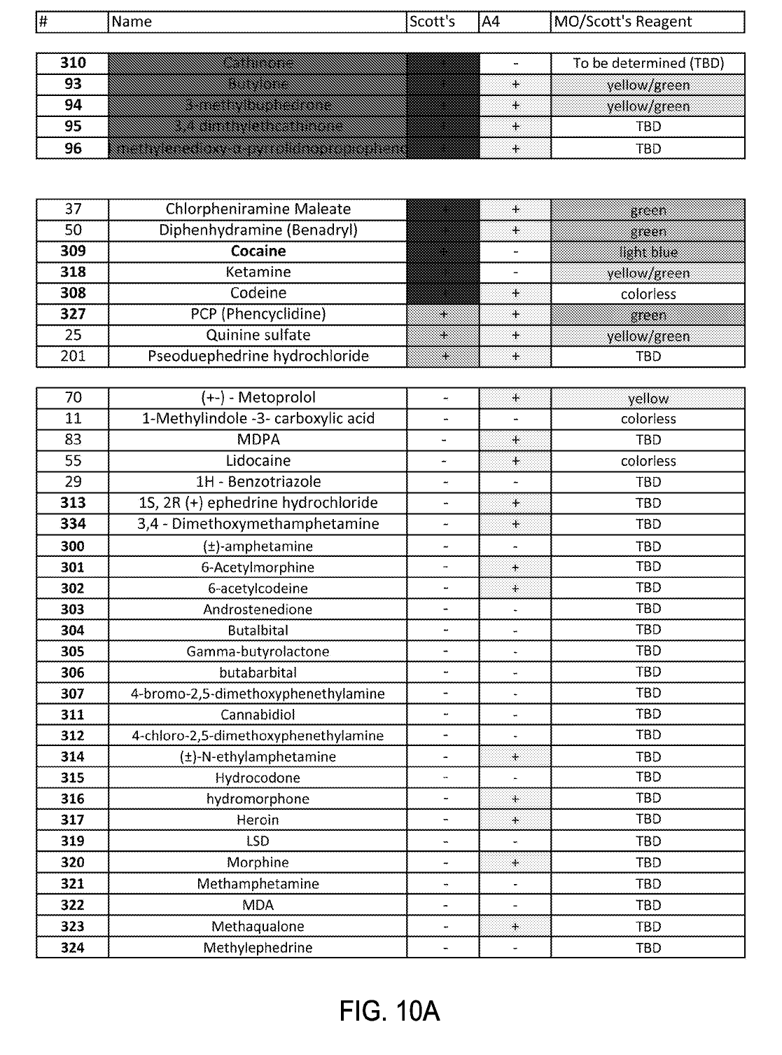

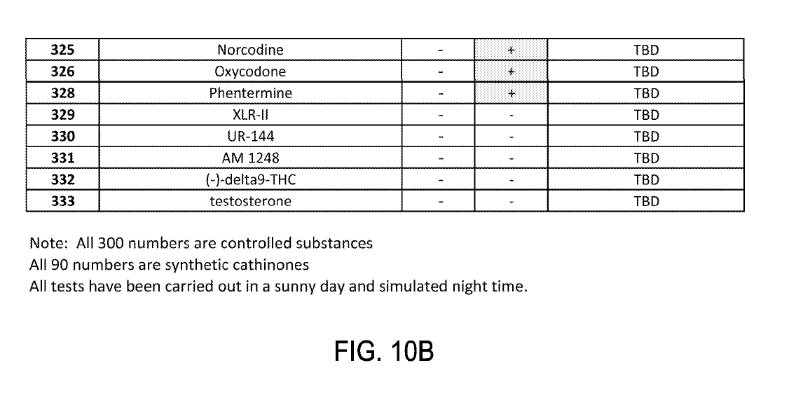

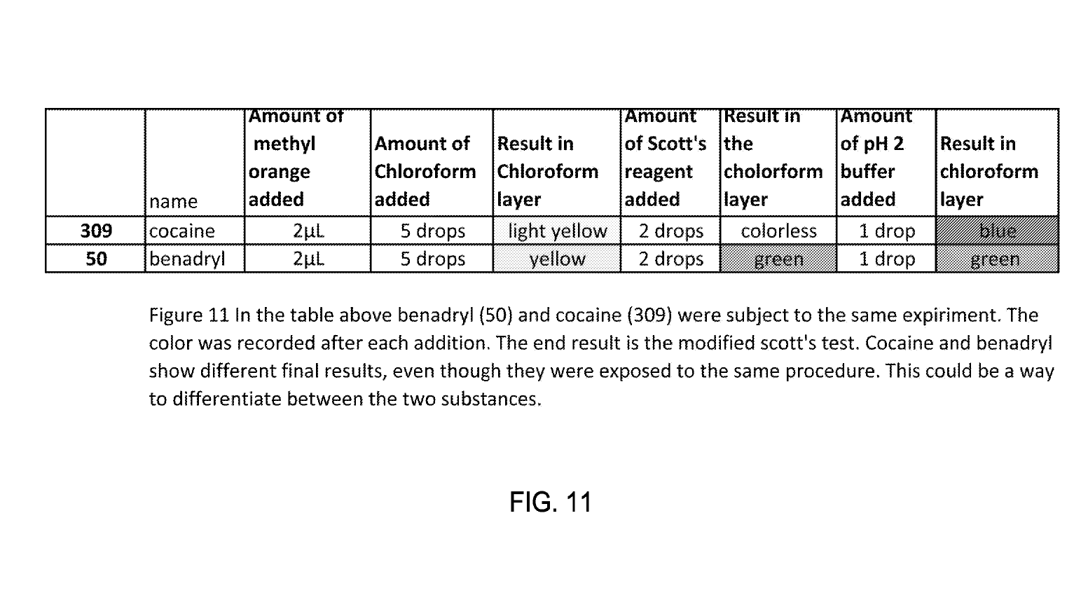

In additional testing, it was found that synthetic cathinones have all positive results at three tested pHs (see FIG. 9). When the pH increases above pH 7, the yellow ion-pair complex will disappear from chloroform layer. A different dye identified the presence of compound #319 (LSD), 322 (MDA), 331 (AM 1248), and 327 (PCP). A combination of that dye and methyl orange offers a way to narrow down the synthetic cathinones.

Further, the reagent in a Scotts test may be combined with methyl orange in one vessel, e.g., an ampoule. That is, mixing methyl orange in the same test tube (or ampoule) with Scott's reagent provides a more powerful, single test. A blue color is observed when cocaine is present which may eliminate several false positives for cocaine found in the current Scott's test. Synthetic cathinones remain yellow in the mixture having methyl orange and Scott's reagent, while many of the false positives in the methyl orange-only test turn green.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A colorimetric assay to detect a piperazine, indoline, indole, azabicyclo, morphinan, or amine containing compound, comprising:
providing a test sample suspected of having a piperazine, an indoline, an indole, azabicyclo, morphinan, or an amine containing compound, and a composition comprising reagents including a buffer, an organic solvent and a sulfonic acid, sultone or oxothian-2,2,dioxide containing dye;
contacting the sample and the composition so as to provide a mixture; and
detecting whether the mixture has a different color than a control mixture that lacks the test sample.

2. The assay of claim 1 wherein the dye is methyl orange, xylenol orange, calmagite, FD&C blue 1, metanil yellow, 1-(2-hydroxyl-1-naphthylazo)-2-naphthol-4-sulfonic acid zinc salt, or 3-((E)-(4-((E)-(4-amino-7-sulfonatonaphthalen-1-yl)diazenyl)-7-sulfonatonaphthalen-1-yl)diazenyl)naphthalene-1,5-disulfonate sodium salt.

3. The assay of claim 1 further comprising contacting a portion of the sample with Dragendorff's reagent or Scott's reagent.

4. The assay of claim 1 wherein the composition comprises a dye other than the sulfonic acid, sultone or oxothian-2,2,dioxide containing dye.

5. The assay of claim 4 wherein the composition comprises Dragendorff's reagent or Scott's reagent.

6. The assay of claim 3 wherein the Dragendorff's reagent comprises bismuth nitrate, potassium iodide, glacial acetate acid and water.

7. The assay of claim 1 wherein the organic solvent is immiscible in water.

8. The assay of claim 1 wherein the buffer has a pH of about 0 to 8.

9. The assay of claim 1 wherein the organic phase of the control sample is clear.

10. The assay of claim 1 wherein the organic phase of the test sample is yellow, blue or red.

11. The assay of claim 1 wherein the dye comprises a sulfonic acid containing dye.

12. The assay of claim 1 wherein the composition is in a receptacle formed of a plastic.

13. The assay of claim 1 wherein the composition is in a receptacle formed of glass.

14. The assay of claim 4 wherein the further dye comprises cobalt thiocyanate.

15. A kit comprising:
a first receptacle having a composition comprising a buffer, an organic solvent, and a sulfonic acid, sultone or oxothian-2,2,dioxide containing dye.

16. The kit of claim 15 further comprising a second receptacle comprising a composition having the buffer, the organic solvent and the dye.

17. The kit of claim 15 wherein the dye is methyl orange, xylenol orange, calmagite, metanil yellow or 1-(2-hydroxyl-1-naphthylazo)-2-naphthol-4-sulfonic acid zinc salt.

18. The kit of claim 15 wherein the composition further comprises a dye other than the sulfonic acid, sultone or oxothian-2,2,dioxide containing dye.

19. The kit of claim 18 wherein the further dye comprises cobalt thiocyanate.

20. The kit of claim 15 wherein the buffer has a pH of about 0 to 8.
